(19) United States
(12) Patent Application Publication
(10) Pub. No.: US 2021/0085809 A1
(43) Pub. Date: Mar. 25, 2021

(54) ENHANCEMENT OF PATHOGEN IMMUNOGENICITY

(71) Applicant: ACADEMISCH ZIEKENHUIS LEIDEN (h.o.d.n. LUMC), Leiden (NL)

(72) Inventors: Fijs W. B. VAN LEEUWEN, Leiden (NL); Meta ROESTENBERG, Leiden (NL)

(73) Assignee: ACADEMISCH ZIEKENHUIS LEIDEN (h.o.d.n. LUMC), Leiden (NL)

(21) Appl. No.: 16/634,221

(22) PCT Filed: Jul. 27, 2018
(86) PCT No.: PCT/NL2018/050527

§ 371 Date: Jan. 27, 2020

(30) Foreign Application Priority Data
Jul. 28, 2017 (NL) 2019373

(51) Int. Cl. A61K 51/04; A61K 49/00; A61K 9/00; A61K 51/06; A61K 51/12; A61K 47/68; A61K 39/39; A61P 31/04; A61K 39/085; A61K 39/015; A61K 47/54; A61P 33/06; A61K 47/61

(52) U.S. Cl. CPC A61K 51/0495; A61K 2039/522; A61K 49/0032; A61K 49/0097; A61K 9/0019; A61K 51/065; A61K 51/1203; A61K 47/6835; A61K 39/39; A61P 31/04; A61K 39/085; A61K 39/015; A61K 47/555; A61P 33/06; A61K 47/61; A61K 49/0054

(57) ABSTRACT

The present invention relates to a vaccine composition for use in in vivo administration, comprising a (attenuated) pathogen or commensal modified to be a pre-targeting vector, the pre-targeting vector comprising one or more pendent reactive moieties able to form a high affinity interaction with a complementary moiety residing on an immunogenic conjugate component.

The present invention relates to methods, compounds, compositions and kits useful for enhancing the immunogenicity of pathogens in an animal or human. Such pathogens may include bacteria, mycobacteria, fungi or parasites.

### BACKGROUND OF THE INVENTION

The efficacy of cancer vaccines or whole organism vaccines, consisting of uni- or multicellular organisms, may be compromised by the natural immune evasive strategies of the pathogen. The use of whole organisms provide an effective means of vaccination because they expose the full panel of antigens to the immune system. However, triggering such an immune response requires large quantities of relatively costly whole organism vaccines. Enhancing the immune response through addition of adjuvants would yield higher responses with reduce vaccine quantities.

Adjuvants may allow for a reduction of the dosage of vaccine required, which is particularly advantageous in large populations in resource-poor settings. One example is the development of attenuated parasite vaccines for malaria. Despite the promising efficacy in human studies, high numbers of sporozoites need to be inoculated intravenously to achieve protective immunity. So far no strategies have been reported that effectively could reduce the numbers of sporozoites required. Also, immediate co-administration of adjuvants with the pathogen may affect the integrity or viability of the pathogen.

Accordingly, it would be desirable to create a vaccine and adjuvant kit that would increase immunogenicity, e.g. stimulate the humoral and cellular responses against poorly immunogenic pathogens in humans or animals. In addition, it would be desirable to create a whole organism vaccine whereby the pathogens, including commensals, are able to distribute to or replicate in their natural niche within the host, which would induce an immune response preferably in the natural local niche of the pathogen or commensal cell. Also, it would be desirable to provide such an enhanced vaccine as a kit of part suitable for pathogenic uni- or multicellular organisms. It would also be highly desirable to provide a method of improving health through preventing or curing infections by pathogens through an in vivo conjugated adjuvant-pathogen vaccine which may be used in healthy humans and animals.

### SUMMARY OF THE INVENTION

Accordingly, the present invention relates to a vaccine composition for in vivo covalent or non-covalent conjugation of a unicellular or multicellular pathogen or commensal cell modified to be a pre-targeting vector, in which the pathogen or commensal cell comprises one or more pendent reactive moieties able to form a high affinity interaction with a complementary conjugate moiety. The complementary conjugate moiety, administered intravenously or locally, will interact with the reactive moiety on the pathogen or commensal cell, increasing its immunogenicity. Hence, the subject invention relates to a two component vaccine composition for use in in vivo administration, comprising a (preferably attenuated) pathogen or commensal (all modified to be a pre-targeting vector), the pre-targeting vector comprising one or more pendent reactive moieties able to form a high affinity interaction with a complementary conjugate moiety residing on an immunogenic secondary component.

In a further aspect, the present invention also relates to an immunogenic adjuvant component for intravenous or local administration and for forming a high affinity interaction with a complementary moiety of the pre-targeting vector composition according to the invention; wherein the adjuvant component comprises at least one agent selected from the group consisting of: a pathogen-associated molecular pattern, antigens, targets for pathogen recognition receptors, adjuvants or a diagnostic agent, an imaging agent, a contrast agent, a therapeutic agent, or a combination or multitude thereof.

The present invention, in a third aspect, also relates to an enhanced vaccine composition comprising a kit of parts in the form of a component that comprises of whole-organism antigens for presentation to a pathogen or commensal organism, and as the second component, a physiologically acceptable component comprising an effective amount of the immunogenic adjuvant.

In a fourth aspect, the present invention also relates to a method of stimulating an immune response in a human or animal against a pathogen or commensal organism, which comprises the steps of a) administering to the human or animal a pathogen or commensal organism (modified to be the pre-targeting vector component), and b) administering to the human or animal a vaccine comprising an immunogenic moiety, inducing or adjuvanting, at the pre-targeted location of the commensal or pathogen, an immune response vis-h-vis the commensal or pathogen (the adjuvant component).

### DESCRIPTION OF THE FIGURES

FIGS. 1 to 6 Relate to Example 1, Illustrating Pre-Targeting Using Supramolecular Interactions:

FIG. 1 Schematically illustrates the concept of a supramolecular the pre-targeting concept according to the invention, i.e. labelling of a S. aureus by functionalising with UBI-Ad₂. This yields a functionalized pathogen (defined as pretargeting vector) that can be administered in step 1. A multimeric cyclodextrin containing polymer (defined as secondary conjugate) further functionalized with diagnostic labels, namely a fluorescent label and/or a ^{99m}Tc-radiolabel can be added in step 2. The combined approach yields a vector: ^{99m}Tc-conjugate complex.

FIG. 2 Shows microSPECT images of mice inoculated with S. aureus-UBI-Ad (encircled location; step 1) for 18 h, followed by the administration of a ^{99m}Tc-labeled multimeric cyclodextrin containing polymer (step 2). Next to some background uptake (stomach and bladder) the figures illustrate that encircled region contain relatively higher quantities of the polymer, indicative for the vector: ^{99m}Tc-conjugate complex and its stability over time.

FIG. 3 Shows the increase in fluorescence intensity in infected tissues when compared to non-infected tissues resected form the mice presented in FIG. 2. To accommodate this read-out, the fluorescent label on the multimeric cyclodextrin containing polymer was used.

FIG. 4 Schematically illustrates the concept of a supramolecular the pre-targeting concept according to the invention, i.e. labelling of a S. aureus by functionalising with ^{99m}Tc-UBI-Ad₂. This yields a functionalized pathogen (defined as pretargeting vector) that can be administered in step 1. A multimeric cyclodextrin containing polymer (defined as secondary conjugate) further functionalized with diagnostic labels, namely a fluorescent label and/or a ¹¹¹In-radiolabel can be added in step 2. The combined approach yields a ^{99m}Tc-vector: ¹¹¹In-conjugate complex.

FIG. 5 Depicts dual-isotope microSPECT image of mice containing ^{99m}Tc-vector (S. aureus-^{99m}Tc-UBI-Ad; encircled) and ¹¹¹In-conjugate. The observed T/NT ratio's for the ¹¹¹In-conjugates as observed in the circles are indicative for the successful formation of the ^{99m}Tc-vector: ¹¹¹In-conjugate complex.

FIG. 6 Depicts dual-isotope microSPECT image of mice containing ^{99m}Tc-control (S. aureus-^{99m}Tc-UBI: encircled) and ¹¹¹In-conjugate. The reduced T/NT ratio's, compared to FIG. 4, for the ¹¹¹In-conjugates as observed in the circles indicate that without the prescence of Ad, Saureus does not act as a vector for complex formation.

FIGS. 7 to 13 relate to example 2, illustrating pre-targeting via click chemistry:

FIG. 7. Schematically illustrates the concept of a the labelling of a S. aureus by functionalising with UBI-Cy5-azide. This yields a functionalized fluorescent pathogen (defined as pretargeting vector) that can be administered in step 1.

FIG. 8. Presents a confocal microscope image showing the A. aureus-UBI-Cy5-azide bacteria as bright fluorescent spots.

FIG. 9 Schematically illustrates the concept of a “click”-chemistry based pre-targeting concept according to the invention, i.e. labelling of a S. aureus by functionalising with UBI-Cy5-azide. This yields a functionalized pathogen (defined as pretargeting vector) that can be administered in step 1. A BCO-functionalized DTPA chelate containing ¹¹¹In (defined as secondary conjugate) can be added in step 2. The combined approach yields a Cy5-vector: ¹¹¹In-conjugate complex.

FIG. 10 Depicts the time dependence of the click reaction between S. aureus-UBI-Cy5-azide and ¹¹¹In-DTPA-DBCO when monitored in vitro. At 3 h post mixing the all the ¹¹¹In-DTPA-DBCO in solution was bound to the bacteria.

FIG. 11. Schematically illustrates the concept of a the labelling of a sporozoite by functionalising with Cy5-azide. Reacting the carboxylic acid group of CY5-azide with primary amines on the surface results in a functionalized fluorescent pathogen (defined as pretargeting vector) that can be administered in step 1.

FIG. 12. Presents three confocal microscope image showing the sporozoited functionalized with Cy5-azide. First row fluorescence images (arrows indicate the location of the banna-shaped sporozoites), second row transmission images, third row overlay of the two.

FIG. 13 Schematically illustrates the concept of click chemistry based pre-targeting concept according to the invention, i.e. labelling of a sporozoites by functionalising with Cy5-azide. This yields a functionalized pathogen (defined as pretargeting vector) that can be administered in step 1. A complementary reactive Cy7 dye (Cy7-DBCO; defined as secondary conjugate) can be added in step 2. The combined approach yields a Cy5-vector: Cy7-conjugate complex.

FIG. 14 This multicomponent image illustrates how Cy5-azide and Cy7-dbco, when they react with each other (A) in solution influence the fluorescence properties of Cy5 (B), resulting in its reduction in fluorescence intensity (quenching).

FIG. 15 Illustrates that reacting Cy7-DBCO to sporozoites functionalized with Cy5-azide, yields a similar quenching effect of the Cy5 fluorescence intensity as was observed when reacting the individual components in solution (FIG. 14).

FIGS. 16 and 17 relate to example 3, illustrating pathogen surface functionalization as a means to alter the interaction with the immune system:

FIG. 16 Schematically presents how early stage malaria parasites, so-called sporozoites can be synthetically modified with proteins, e.g. using antiCSP antibodies (A). In addition it presents that this functionalization results in preferred uptake by immune cells (B).

FIG. 17 Shows the enhanced recognition of the above modified SPZ by immune cells, in more detail: Monocyte-derived dendritic cells and macrophages were incubated with genetically modified Plasmodium berghei sporozoites expressing GFP with or without antiCSP antibodies for one hour. Uptake of fluorescent sporozoites was measured by flow cytometry.

### DETAILED DESCRIPTION OF THE INVENTION

The present invention relates to a novel vaccine method or kit, and its application in methods of treatment and creation of an immune response in humans. Preferably the pathogen against which the vaccine is directed is a bacterium, or protozoan or multicellular parasite which may be located intra- or extracellularly.

The present invention ultimately permits to alter the surface of functionalised pathogens or commensals in situ with an immune-enhancing agent, thereby resulting in an increased or stronger immune response.

The term “immunogen” refers to a substance which is capable, under appropriate conditions, of inducing a specific immune response and of reacting with the products of that response (e.g., a specific antibody, adjuvants, ligands to pathogen recognition receptors, antigens, specifically sensitized T-lymphocytes or a combination thereof).

As used herein, the term “pathogen” refers to a disease-causing unicellular or multicellular organism that is acting as a pathogen of a human or animal. Animals may include mammals in general, livestock, cows, sheep, pigs, monkeys, dogs, cats, rats, arthropods, birds, reptiles, fish, and insects.

“Attenuated” herein relates to a pathogen with reduced virulence, which may be alive or dead, metabolically active or non-active. The pathogen or infectious agent has been altered or chosen such that it is harmless or less virulent or less reactogenic, which is a well-described technology in vaccine development.

As used herein, “microbe” denotes bacteria, rickettsia, mycoplasma, algae, protozoa, fungi and like microorganisms, e.g. malaria parasites, spirochetes and the like, “parasite” denotes infectious, generally microscopic or very small multicellular invertebrates, or ova or juvenile forms thereof, which are susceptible to antibody-induced clearance or lytic or phagocytic destruction, e.g. amoeba, helminths and the like, while “infectious agent” or “pathogen” denotes both microbes and parasites.

The term “commensal” herein refers to a commensal of a human or animal host. Commensal microbes are usually part of the human or animal intestinal, or skin microbial flora. Typically, commensal microbes, in particular bacteria have co-evolved with their host to provide nutrients, protect against pathogens, and aid in intestinal development, as applicable. Commensal microbes include, but are not limited to one or more bacterial selected from the genera Adlercreutzia, Oscillopira, Mollicutes, Butyrivibrio, Bacteroides, Clostridium, Fusobacterium, Eubacterium, Ruminococcus, Peptococcus, Peptostreptococcus, Bifidobacterium, Rikenella, Alistipes, Marinilabilia, Anaerostipes, Escherichia, and/or Lactobacillus.

The terms “conjugate”, “conjugate moiety” and “conjugate component” are user exchangeably herein, and refer to the secondary component that is modified to selectively attach to the pre-targeting vector.

In a first aspect of the invention, a targeting cell coated with the primary moiety (pre-targeting vector) is administered in vivo; upon target localization of the (optionally attenuated) pathogen or commensal, a covalent or non-covalent immune-inducing conjugate moiety is administered intravenously or locally.

This two-step method not only provides targeting of the immune-inducing conjugate moeity, but also may induce internalization of the subsequent vector: conjugate complex on the pathogen or commensal into the target cell, which may be an antigen-presenting cell.

Alternatively, another embodiment provides a three-step protocol that produces a pre-targeting vector: conjugate: secondary conjugate complex at the surface, wherein the secondary conjugate is administered simultaneously or within a short period of time after administration of primary conjugate, preferably before the vector-conjugate complex has been removed from the target cell surface. Additional internalization methodologies are contemplated by the present invention and are discussed herein.

The present invention thus makes use of at least two components, the so-called pre-targeting vector and the conjugate component, whereby one is first locally accumulated, and the second component is then introduced into the patient, to selectively couple covalently or non-covalently to the component already in place.

The two components (vector and conjugate component) provide complementary functionality. The term “complementary functionality” herein refers to a highly selective binding chemistry, wherein two or more complementarily functionalised partner molecules are likely to react, or bind in a predetermined reaction pathway, covalent or non-covalent. Once engaged, the two components form a vector: conjugate complex or vector: conjugate matrix.

As stated, the vector: conjugate recognition may advantageously be done using selective physical interactions, such as those provided in supramolecular host-guest inclusion complexes.

A preferred example for such inclusion complexes are for instance an adamantane (Ad) as host moiety, and a cyclodextrin (CD) as guest moiety. Alternatively, or additionally, a vector conjugate recognition may also be done by selective covalent chemistry, e.g. chemical bonding as for instance using “Click” chemistry between azide and alkyne moieties, whereby the two reactants advantageously are a moiety bound to the vector compound and a moiety bound to the conjugate, which will react to form a covalent bond when exposed to each other under appropriate conditions.

The present invention, both compositions and method have the clear benefit of providing a first introduction of a pre-targeting vector that in itself may not need to have any strong immunogenic, therapeutic or diagnostic effect, to verify the accurate location, and stability of the positioning of the vector at this location, and then to modify the vector in situ using a secondary conjugate component functionalized with one or more desired functionalities. This allows for the natural distribution or targeting of the pathogen, including unaffected viability, whereas the conjugation step with addition of the immunogen can be performed at a later stage or locally in preferred organs only. In one aspect the present invention provides a pharmaceutical composition useful as a vaccine, comprising an attenuated pathogen or commensal and an effective amount of the adjuvanting conjugate moiety, the resulting composition capable of eliciting the vaccinated host's cell-mediated immunity for a beneficial response.

In another aspect, the invention provides for a composition comprising an attenuated pathogen. This pre-targeting vector may be employed directly administered with, or close in time to, the conjugate component.

In another aspect, the invention provides a method for preparing a vaccine composition containing an attenuated pathogenic microorganism, comprising or chemical alterations to a pathogen or commensal to form a pre-targeting vector, preferably with an enhanced ability to elicit the vaccinated host's immune response, by adding to the vaccine composition an effective amount of the immunogenic conjugate component.

Malaria caused by Plasmodium spp is an infectious disease of public health importance. The most severe forms of malaria are usually caused by Plasmodium falciparum; and control of the parasite and/or the mosquito vector is vital for disease prevention and elimination. The sporozoite stages of P. falciparum are the first stages of the parasite to be exposed to the host immune response and are vulnerable because of their extracellular location.

Circumsporozoite protein (CSP) is the most abundant sporozoite antigen that is relatively more conserved compared to merozoite surface antigens. A vaccine comprising of CSP antigen is currently registered for human use, but induced protection is partial and wanes over time. Enhancement of anti-CSP immune responses in the context of other sporozoite antigens is desirable. Without wishing to be bound to any particular theory, it is believed that a two-step vaccination approach in which the sporozoites are allowed to enter the hepatic cells and express antigens which are crucial for the induction of cellular immune responses while in a second step increasing the immunogenicity of extracellular sporozoites by covalently or non-covalently conjugating immunogens to the remaining attenuated P. falciparum sporozoites, will increase the immune response, thereby leading to a much stronger immunity at a much lower rate of inoculation as compared to the non-conjugated sporozoite vaccine.

Accordingly, modifying sporozoite surfaces in vitro, and subsequently targeting the attenuated pathogen to antigen presenting cells was found to increase internalisation of sporozoites, making the sporozoites visible to the immune system, and possibly increasing the effect of vaccination.

Concentration of the Pre-Targeting Vector

The amount of the pre-targeting vector as well as the conjugate component to be administered depends on the immune response achieved. Typically, the number of pathogens or commensals modified to be pre-targeting vectors, to be injected into the vascular system and/or tissue of a patient should be sufficient in order to achieve an optimal balance between antigen presentation, while observing the tolerability and safety for the human or animal.

While the number of pathogens or commensals which may be injected or otherwise delivered will vary depending upon the patient's metabolism, body weight, organ weight, etc., it has been found that approximately 200 to 2,000,000,000, optionally attenuated pathogens suspended in a biologically safe solution and delivered to the patient may suffice, although larger quantities of pathogens may be necessary and desirable depending on various circumstances and conditions of use.

By knowing the concentration of pathogens in a given volume of dispersion, one merely needs to withdraw and inject the desired volume of liquid containing the desired number of pathogens. This obviously also applies to commensals.

The present invention makes use of at least two components, a pre-targeting vector, also referred to herein as the primary component, and at least a first conjugate, also referred to herein as the secondary component.

Primary and secondary components are functionalised to provide complementary functionality. The term “complementary functionality” herein refers to a highly selective binding chemistry, wherein two or more complementarily functionalised partner molecules are likely to react, or bind in a predetermined reaction pathway.

As stated, the vector-conjugate recognition may advantageously be done using selective physical interactions, such as those provided in supramolecular host-guest inclusion complexes.

A preferred example for such inclusion complexes are for instance an adamantane (Ad) as primary moiety, and a cyclodextrin (CD) as secondary moiety.

Alternatively, or additionally, a vector-conjugate recognition may also be done by selective covalent chemistry, e.g. chemical bonding as for instance using “click” chemistry between azide and alkyne moieties, whereby the two reactants advantageously are a primary moiety bound to the vector compound, and a secondary moiety bound to the conjugate, whereby the vector and conjugate will react to form a covalent bond when exposed to each other under appropriate conditions.

The present invention, both compositions and method, have the clear benefit of providing a first allowing to introduce a pre-targeting vector that in itself may not need to have any strong immunogenic, therapeutic or diagnostic effect, to verify the accurate location, and stability of the positioning of the vector at this location, and then to modify the vector in situ using a secondary component functionalized with one or more desired activities. This may reduce for instance the number of vaccine injections required for a suitable immune response, but may also reduce the amount of pathogen (vector) components. Also, other diagnostic or therapeutic activities may be coupled with the vector component or the conjugate that so far were not accessible or possible.

One aspect in the present invention provides a 2-component pharmaceutical composition useful as a vaccine, comprising an optionally attenuated and host-labelled pathogenic microorganism, and an effective amount of the adjuvanting conjugate moiety, the resulting composition capable of eliciting the vaccinated host's humoral or cell-mediated protective immune response to the pathogen.

In another aspect, the invention provides for a composition comprising an attenuated and labelled pathogenic microorganism. This pre-targeting vector composition may be employed directly, and administered with, or close in time to, the adjuvanting conjugate component.

In another aspect, the invention provides a method for preparing a vaccine composition containing an attenuated pathogenic microorganism, comprising labelling a pathogenic microorganism to form a pre-targeting vector, preferably with an enhanced ability to elicit the vaccinated host's immune response against the pathogen, by adding to the vaccine composition an effective amount of the immunogenic conjugate component, a subunit, or a biologically active fragment thereof.

The pre-targeting vector preferably comprises pathogens or commensals which are allowed to distribute to and/or replicate in their natural (local) niche, where they may be extracellular in the interstitial cell fluid, on mucosal surface or in the microvascular bed, or diffuse in the lymphatic network of tissue or may accumulate in cells (e.g. macrophages). As such, these pathogens or commensals keep their natural tropism towards specific organs.

The pre-targeting vector pathogens according to the present invention may preferably also comprise an imaging label, e.g., a diagnostic and/or a detectable label. This advantageously permits to determine if and when the pre-targeting vector pathogens are in the desired location, and of any loss occurs due to blood flow or degradation that may negatively impact a subsequent treatment.

Vector and Conjugate Functionality

The terms “vector” and “conjugate” as used herein refer to two different, but complementary binding partners that non-covalently, or covalently interact with each other. As used herein, the term “vector moiety” or “group” means the part or moiety of a monomer of the vector molecule, which enables the covalent or non-covalent binding to a complementary conjugate functional group.

A “conjugate molecule” is in turn a molecule that comprises one or more functional groups, where a monovalent conjugate molecule comprises one conjugate functional group and a multivalent conjugate molecule comprises at least two conjugate functional groups. As used herein, the term “conjugate functional group” means the part or moiety of a monomer of the conjugate molecule, which enables the covalent or non-covalent binding to a complementary vector functional group. A “vector molecule” is in turn a molecule that comprises one or more vector functional groups, where a monovalent vector molecule comprises one vector functional group and a multivalent vector molecule comprises at least two vector functional groups.

Preferably, one conjugate moiety specifically interacts with a matching vector moiety. Where non-covalent vector-conjugate pairs are employed, according to the present invention, the interaction between the conjugate and the vector may be reversible, and is determined by the affinity strength as expressed by dissociation constants. Typically a conjugate molecule does not normally interact with another conjugate molecule.

Examples for a non-covalent vector-conjugate interaction include beta-cyclodextrin-adamantane, beta-cyclodextrin-ferrocene, gamma-cyclodextrin-pyrene, cucurbituril-viologen, and/or a Ni(NTA)-His tag.

Examples for a covalent interaction include an Azide (N₃)— alkyne interaction as a vector-conjugate interaction, such as for instance those providing metal-free bioorthogonal cycloadditions between strain-promoted alkynes, so called cyclooctynes, with azides (SPAAC), tetrazines or nitrones (SPANC).

For example, the dibenzocyclooctyne group (DBCO) or bicyclo[6.1.0]nonyne (BCN) allowcopper-free “Click” chemistry to be applied to vectors to be used in live organisms. DBCO or BCN groups will preferentially and spontaneously label molecules containing azide groups (—N₃). Also, within physiological temperature and pH ranges, the DBCO or BCN group does not react with amines or hydroxyls naturally present in many biomolecules. Also, the reaction of the DBCO or BCN group with the azide group is significantly faster than with sulfhydryl groups, making this a highly selective reaction. Other suitable “click” materials may be used as well.

Preferably, the reaction affinity and reaction speed of the conjugate component are high to allow for rapid in vivo binding to the pre-targeting vector. Accordingly, potential covalent binding moieties for the pre-targeting vector and secondary conjugate component preferably will be tested prior to use, to select the desired functional compounds.

The term “inclusion complex” herein refers to any material wherein the vector compound absorbs or embeds a conjugate compound to form a complex. For example, the conjugate compound may be embedded in a cavity formed by the vector compound.

Preferably, the vector-conjugate components are each readily available, and enable to perform the methods of the present invention on a relatively large scale and/or in low cost devices.

As used herein, the term “vector-conjugate molecule interactions” includes the non-covalent binding between respective conjugate and vector functional groups. In a preferred setting, hydrophobic interactions, such as lipophilic interactions, are being used instead of interactions that are based on charge.

The functionality may be reversed, i.e. a conjugate functional group may be linked to the pre-targeting vector, and a vector functional group to the conjugate component. The choice for a suitable vector or conjugate modification largely depends on the effect that such vector or conjugate, including the way it is attached or bound, may have on the ability of the components to perform their tasks.

Also, multivalent vector and multivalent conjugate structures may be employed, comprising multiple vector and/or conjugate functional groups. In such case, some of the vector functional groups may be (non-) covalently bound to a conjugate molecule, while others remain free, or vice versa.

Preferred, essentially non-covalent vector moieties or compounds are cyclodextrins, with adamantane moieties acting as conjugate molecules.

Cyclodextrins are cyclic polysaccharides containing naturally occurring D(+)-glucopyranose units in an α-(1,4) linkage. The most common cyclodextrins are alpha (α)-cyclodextrins, beta (β)-cyclodextrins and gamma (γ)-cyclodextrins which contain, respectively, six, seven or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other.

Preferably beta-cyclodextrin is used, which is the best binding partner for adamantane. The cyclodextrin may contain additional groups, such as an amine to attach it to a scaffold, one or more thiols to bind the cyclodextrin to a gold surface, or hydroxypropyl groups to increase solubility and biocompatibility. Other members of the cyclodextrin family (most likely alpha and gamma) can also be used for vector-conjugate interaction, although different conjugates have to be introduced to achieve this.

Accordingly, a good, but not the only, example of supramolecular vector-conjugate interactions that can be applied in the invention is the non-covalent interaction between adamantane (as the vector molecule) and 3-cyclodextrin (as the conjugate molecule). The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Various cyclodextrin containing polymers and methods of their preparation are also known in the art. (Comprehensive Supramolecular Chemistry, Volume 3, J. L. Atwood et al., eds., Pergamon Press (1996)). A process for producing a polymer containing immobilized cyclodextrin is described in U.S. Pat. Nos. 5,608,015, or 5,276,088 describe methods of synthesizing cyclodextrin polymers by either reacting polyvinyl alcohol or cellulose or derivatives thereof with cyclodextrin derivatives or by copolymerization of a cyclodextrin derivative with vinyl acetate or methyl methacrylate. The resulting cyclodextrin polymer contains a cyclodextrin moiety as a pendant moiety off the main chain of the pathogen or commensal cell. Cyclodextrin-based polymers have been used for therapeutic applications (Kandoth et al. Two-photon fluorescence imaging and bimodal phototherapy of epidermal cancer cells with biocompatible self-assembled polymer nanopathogens. Biomacromolecules 2014 (15):1768-1776) and imaging agents (Yan et al. Poly beta-cyclodextrin inclusion-induced formation of two-photon fluorescent nanomicelles for biomedical imaging. Chemical Communications 2014 (50):8398-8401) and they showed excellent biocompatibility. Adamantane is a lipophilic small molecule that may be attached to a vector that can be physically lodged in the target tissue. The adamantane structure combines rigidity with the ability to form diamondoid structures, and offers high binding affinities with cyclodextrins. In one aspect, the conjugate molecule may be adamantane, whereas the vector molecule is preferably then a cyclodextrin that non-covalently interacts with adamantane. Both compounds are relatively cheap and easy to produce in controlled settings and non-toxic.

Scaffolding

The vector moiety may be connected to the pathogen by a “scaffold”, i.e. a binding unit comprising a spacer molecule or otherwise suitable molecular structure. The scaffold may be intended to ensure that the vector moiety is presented to the incoming conjugate moiety, and/or may permit to modify the pathogen or commensal cell easily. Also, several vector moieties or vector functional groups may be interconnected through a scaffold molecule to form a multivalent vector structure. The term “multivalent” as used herein refers to a number of vector or conjugate molecules, or functional groups thereof, that are part of the same molecule or structure.

Multivalent interactions contain at least two functional groups of the same type (e.g. at least two vector functional groups, or at least two conjugate functional groups) bound to each other through a backbone (or scaffold) that allows the multimerization of the matching vector or conjugate molecule. The upper limit in multivalency depends on the effectiveness of performing the desired functionality of the pathogen or commensal, e.g. whether the cell still is alive, and can interact with other cells or possibly replicate. Without wishing to be bound to any particular theory, it is believed that multivalency enhances the affinity, and thus improves the binding, as monomeric vector molecules tend to show a significantly lower noncovalent interaction.

According to the present invention, a “multivalent vector structure” or a “multivalent conjugate structure”, is a structure comprising at least two vector functional groups or conjugate functional groups, respectively. It can in principle be a dimer or polymer of suitable vector or conjugate monomers, but typically the vector or conjugate molecules have been attached or engrafted onto a polymer of a different type that allows for the attachment of the vector or conjugate molecules.

In one embodiment of the present invention, a “multivalent vector structure” or “multivalent conjugate structure” preferably comprises a scaffold or linker structure onto which the at least two vector molecules or at least conjugate molecules have been attached or engrafted resulting in that the scaffold structure comprises at least two vector host functional groups or at least two conjugate functional groups. The scaffold structure can be anything that allows attachment of the vector or conjugate molecules of choice. The scaffold structure may of course also be part of the pathogen or commensal cell.

In another embodiment, the scaffold molecule may be an antibody or polypeptide comprising less than about 30, such as, e.g. less than about 25, less than about 20, less than about 15, less than about 10, less than about 5 or less than about 6 amino acids. It may also be an oligo peptide such as, e.g. a dipeptide, a tripeptide, a tetrapeptide or a pentapeptide. In one embodiment the repeat unit of the poly or oligepeptide is β-alanine.

Conjugation Component

The conjugate components according to the present invention tend to have a smaller size than the pre-targeting vectors, and can be tuned for their pharmacokinetics. This way retention of the conjugate component at the surface of the pre-targeting vector can be combined with a low degree of background accumulation.

Conjugates may be based on synthetic, or naturally occurring compounds, or combinations thereof. Preferably, for covalent interactions with the vector moiety, the conjugate component may be formed of monomeric materials, for instance those provided for click chemistry. Examples for a covalent interaction include an Azide (N₃)— alkyne interaction, for example, the dibenzocyclooctyne group (DBCO) or bicyclo[6.1.0]nonyne (BCN) allow copper-free “Click” chemistry. While this was shown for diagnostic agents (both fluorescent and a radioisotope containing chelates), it is noted that any mono or multimeric functionality including immunogenic or therapeutic moieties and likes will bind to the pathogen surface following straight forward attachment of a DBCO or BCN unit to the agent.

Preferred, essentially non-covalent vector moieties or compounds are cyclodextrins, with adamantane moieties acting as conjugate molecules. Alternatively, for non-covalent interaction with the vector moiety, the conjugate materials may be formed of polymeric materials such as, aminoacid sequences, polylactic acid, polyglycolic acid, polycaprolactone, polystyrene, polyolefins, polyesters, polyurethanes, polyacrylates and combinations of these polymers, and homo-or copolymers, and blends thereof. The particulate materials may further comprise suitable binding agents such as gelatin, polyethylene glycol, polyvinyl alcohol, 16ydroxyl16, (poly)saccharides, DTPA, DOTO, NOTA other hydrophilic materials, and combinations of these. Suitable gelatins may include bovine collagen, porcine collagen, ovine collagen, equine collagen, synthetic collagen, agar, synthetic gelatin, and combinations of these. Examples of useful synthetic polymers formed by chemical cross-linking include polyethylene glycol (hereinafter, “PEG”), polypropylene glycol (hereinafter, “PPG”), polyvinyl alcohol (hereinafter, “PVA”), polyacrylic acid, hydroxyethyl acrylate, polyhydroxy ethyl methacrylate, polyvinyl pyrrolidone, carboxymethyl cellulose, dextrans or other sugar-based polymers; homopolymers or (block) copolymer or the water-soluble polymer of the water-soluble polymer selected from the group consisting of hydroxymethyl cellulose and hydroxyethyl cellulose, α-hydroxyl acids, the α-hydroxyl acid cyclic dimer, a block copolymer of a monomer selected from the group consisting of hydroxyl dicarboxylic acids and cyclic esters may be employed.

Partially water-soluble polymers may be used, wherein typically biocompatibility is higher than for hydrophobic polymers. Also, due to the presence of hydroxyl groups, PEG, PPG, PVA, poly hydroxyethyl acrylate, poly hydroxyethyl methacrylate (hereinafter, “poly-HEMA”) permits easy functionalization. Particularly useful hydrogel beads are those made from polyhydroxy polymers such as polyvinyl alcohol (PVA) or copolymers of vinyl alcohol, which may be readily tagged with the conjugate forming moiety by reaction of pendent hydroxyl moieties within the polymer network, using for instance activating agents such as carbonyldiimidazole.

The weight average molecular weight of useful conjugate components, is preferably 200 or more. Further, for a discharge ex vivo to be facilitated by the living body, it is preferably 50,000 or less. The weight average molecular weight of the conjugate component may be suitably selected such that the desired pharmacokinetics are achieved, e.g. the minimal non-specific binding is surpassed. The weight average molecular weight of the polymers employed may be advantageously determined by gel permeation chromatography.

The conjugate selectively links up or react with the pre-targeting vector comprising the vector moiety. The selectivity of this linkage preferably allows to introduce the conjugate intravenously, rather than into the tissue, as the conjugate then automatically binds to the vector when present at multiple locations, but may also be given locally at the site where the vector was deposited.

Also, several different components may be administered, allowing for a combination therapy. The conjugate may comprise a conjugate moiety or molecule which may react with or bind to a vector structure, and at least a first immunogenic agent. Also, various other agents may be coupled to the conjugate, e.g. diagnostic or imaging labels, or agents that carry a further function.

An exemplary method for preparing a modified vector or conjugate may include providing a pharmaceutically acceptable polymer, and coupling, e.g., by coating, covalent linkage, or co-localization, to the surface of the pre-targeting vector or conjugate, and separately coupling the immunogenic agent, and an imaging agent, a detectable label, or otherwise functional moiety.

The method may further include forming one or more conjugate suspensions, passing the conjugate suspension through a filter, removing impurities from the conjugate suspension, centrifugation to pellet the conjugates, dialyzing the conjugate suspension, and/or adjusting the pH of the conjugate suspension. The method may also include quenching the covalent linking reaction.

Suitable conjugate components may include organic or inorganics components or mixtures thereof. For example, the conjugates can be selected from polymers. Suitable polymers for example can be selected from poly(isobutylene-alt-maleic anhydride) (PIBMA), PAMAM, poly-acrylic acid, polysaccharides, polypeptides and oligopeptides. Some such polymers, such a PIBMA, have the further advantage that they prevent interaction with the immune system and thereby also function as a cloaking group until the immunogenic agent is to be revealed.

For many applications, it is also of importance that the selected structure is preferably non-toxic/immuniogenic on its own. Suitable polymeric conjugates may comprise a pharmaceutically acceptable polymer core and a one or more immunogenic agents and/or imaging agents.

Preferably, the conjugate may comprises a pharmaceutically acceptable polymer core, and one or more bioactive agents, such as a drug or medicament encapsulated in the core, or an antibiotic.

Most importantly, though, the conjugate, the multivalent exposure of the conjugate component on the cell/pathogen surface, or a different effect needs to elicit, adjuvant or polarize an immune response, and hence may induce a therapeutic or prophylactic effect. This preferably achieved by including one or more immunogenic agents on or in the vector:conjugate complex. Such an “immunogen” refers to a substance which is capable, under appropriate conditions, of inducing a specific immune response and of reacting with the products of that response, e.g., a specific antibody, specifically sensitized T-lymphocytes or both, while the term “immunogenic” relates to a reaction triggered by the presence of the immunogen. Immunogenic, or immune response enhancing agents may include, but are not limited to a pathogen-associated molecular pattern, an antigen, and/or a target for pathogen recognition receptors or adjuvants.

Immunogenic agents according to the invention may preferably thus include but are not limited to, a nucleic acid, DNA (a vector or plasmid), an RNA (e.g., an mRNA, the transcript of an RNAi construct, or a siRNA), a small molecule, a peptidomimetic, a protein, peptide, glycan, lipid, surfactant and combinations thereof.

Administration of the Components

Administration of the Pre-Targeting Vector

The vector components according to the present invention may be administered intravenously, locally and orally, i.e. they are injected or infused into a particular area, and/or into a particular organ or tissue in a patient body. Examples include intradermal, subcutaneous, intramuscular and/or intranasal injections or infusions.

The conjugate composition may be chosen and designed in among other factors size, size distribution, compressibility, water content, flowability, deformation creep and/or stability, as well as optimal pharmacokinetics, e.g. rapid clearance and minimal background of non-complexed components, such that they can be infused or injected.

For local enhancement, the delivery of the pre-targeting vector via injection or implantation provides a means to effectively target the vector to its specific natural niche or location, thereby ensuring that the immune response is induced at the site where effector immune responses are most needed.

Moreover, the administration of the vector material via implant or needle based injection can usually be performed on an outpatient basis, resulting in a lower cost than other surgical forms. In particular live, optionally attenuated, pathogens may be simply injected or otherwise delivered, such that the pathogen can distribute to or replicate in its natural niche.

Preferably, “delivering” comprises positioning a delivery device, e.g. a syringe or infusion catheter in proximity to a target region of a blood vessel, or directly into a target tissue not via vasculature, and ejecting the pathogens from the delivery device such that the pathogens are positioned in the target region. In addition, delivery may be accomplished transdermally or transmucosally by a spray, cream, plaster or oral paste or solution.

Administration of the Conjugation Component

The conjugate preferably is administered intravenously, or more generally intravascularly, since the components are accumulated at the pre-targeting vector component location, or likely excreted if not bound to the vector. Alternatively, the conjugate may be injected locally, e.g. by inserting a cannula into the desired region, and injecting the material into the vicinity of the vector component, which includes intradermal, intramuscular, subcutaneous, transdermal or transmucosal administration.

Administration Medium

Components according to the present invention are preferably dispersed in an appropriate dispersion medium. The administration medium of the components may be a buffer/serum solution, and may further comprise injection dispersing agent such as polyoxyethylene sorbitan fatty acid ester or carboxymethyl cellulose, such as methyl paraben or propyl paraben preservatives, sodium chloride, preservatives used in tonicity agents or injections, such as mannitol or glucose, stabilizer, solubilizing agents or excipients.

The present invention thus preferably relates to a primary vector component for in vivo administration and for forming the selective non-covalent high affinity interaction with a complementary secondary conjugate moiety, and/or the selective covalent bond with a secondary conjugate compound having a complementary functionality of the vector component according to the invention; wherein the component comprises a complementary functionality, and at least a first diagnostic and/or therapeutic agent. Preferably, the agent is selected from the group consisting of: a diagnostic agent, an imaging agent, a contrast agent, and a therapeutic agent, preferably a radioactive isotope or a chemotherapeutic drug.

Preferably, the agent is selected from the group consisting of one or more: anti-cancer agents, antibiotics, antihistamines, hormones, steroids, therapeutic proteins, biocompatible materials, imaging agents and contrast agents.

Preferably, the diagnostic agent is selected from the group consisting of magnetic resonance contrast agents, radioopaque contrast agents, ultrasound contrast agents, fluorescence dyes and nuclear medicine imaging contrast agents, more preferably, wherein the radioactive isotope is selected from the group consisting of ^{99m}Tc, ¹¹¹In, ⁸⁹Zr, and/or ⁶⁸Ga

The present invention also relates to a method, and compound of use for locally treating a disease, comprising administering to a target area of a patient in need thereof a kit according to the invention suitable for diagnosing and treating the disease.

The present invention also preferably relates to an enhanced vaccine composition comprising an immunogenic amount of a component that presents antigens from a pathogen or commensal, and a physiologically acceptable adjuvant vehicle comprising an effective amount of the immunogenic adjuvant.

The present invention also preferably relates to a method of stimulating an immune response in a human against an infectious agent or pathogen, which comprises the steps of: (a) administering to the human a pathogen or commensal (the pre-targeting vector component), and (b) administering to the human a physiologically acceptable vaccine vehicle comprising label-specific binding partner comprising an immunogenic moiety, inducing or adjuvanting, at the location of an infectious agent, an immune response vis-à-vis the infectious agent (the adjuvant component).

Preferably, the method further comprises permitting the pre-targeting vector to reach the desired location before inducing or adjuvanting the immune response.

Preferably, the agent is an infectious microorganism selected from the group consisting of bacteria, rickettsia, mycoplasma, protozoa, helminths and fungi.

In one embodiment, the human or animal is suffering from infection by an infectious microorganism selected from the group consisting of bacteria, rickettsia, mycoplasma, protozoa and fungi, or an infectious parasite. In a different embodiment, the human or animal is not suffering from an infection, and wherein the immune response results in protective immunity against infection by an infectious agent exhibiting a targeted infectious agent marker, or against microbiome disturbances by inducing immunity against specific commensals.

Preferably, the method further comprises a secondary conjugate which may be administered simultaneously or within a short period of time after administration of primary conjugate, to form an in-situ complex comprising the pre-targeting vector: conjugate: secondary conjugate. Preferably, such secondary conjugate is administered before a pre-targeting vector: conjugate has been removed from a target cell surface.

The following, non-limiting examples illustrate the invention.

### Example 1 Illustrates Pre-Targeting Using Supramolecular Interactions; Example 2, Illustrates Pre-Targeting Via Click Chemistry, and Example 3 Illustrates Pathogen Surface Functionalization as a Means to Alter the Interaction with the Immune System

All chemicals were obtained from commercial sources and used without further purification. NMR spectra were obtained using a Bruker DPX 300 spectrometer (300 MHz, ¹H NMR) or a Bruker AVANCE III 500 MHz with a TXI gradient probe. All spectra were referenced to residual solvent signal or TMS. HPLC was performed on a Waters system by using a 1525EF pump and a 2489 UV detector. For preparative HPLC a Dr. Maisch GmbH, Reprosil-Pur 120 C18-AQ 10 μm column was used and a gradient of 0.1% TFA in H₂O/CH₃CN (95:5) to 0.1% TFA in H₂O/CH₃CN (5:95) in 40 min as employed. For analytical HPLC a Dr. Maisch GmbH, Reprosil-Pur C18-AQ 5 μm (250×4.6 mm) column was used and a gradient of 0.1% TFA in H₂O/CH₃CN (95:5) to 0.1% TFA in H₂O/CH₃CN (5:95) in 40 min was employed. MALDI-ToF measurements were performed on a Bruker Microflex. High resolution mass spectra were measured on an Exactive orbitrap high-resolution mass spectrometer (Thermo Fisher Scientific, San Jose, Calif.) and processed with the use of Thermo Scientific Xcalibur software (V2.1.0.1139). For dialysis Sigma Pur-A-Lyzer™ Mega 3,500 units were used.

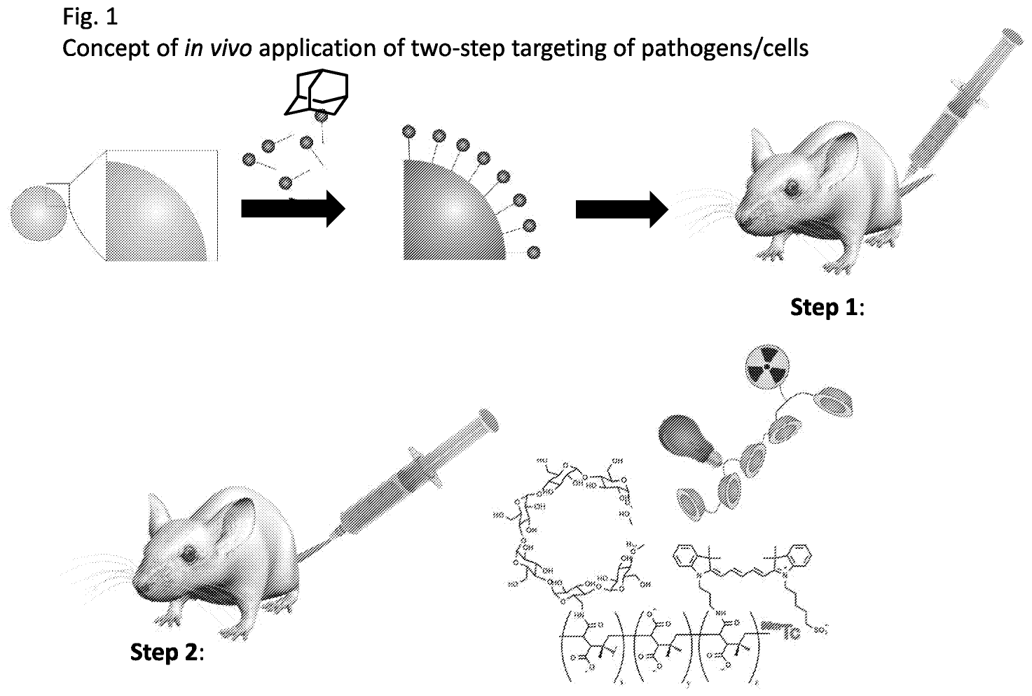

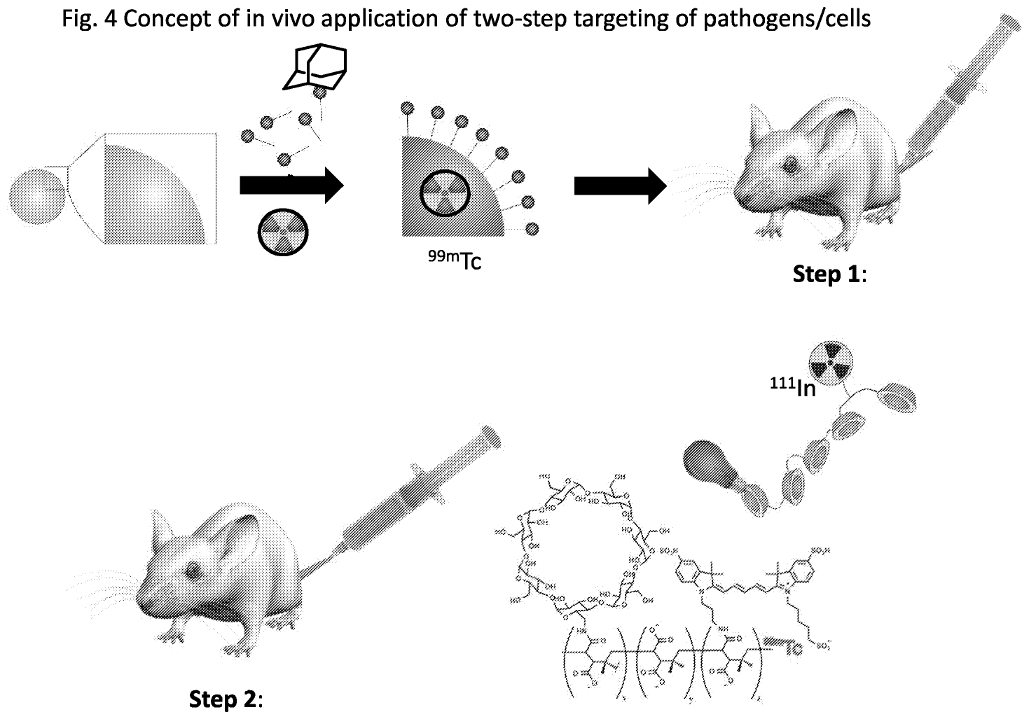

### Example 1: In Vivo Pre-Targeting of Functionalized Staphylococcus aureus (See Also FIGS. 1 and 4, Illustrating the Concept of In Vivo Application of Two-Step Targeting of Pathogens/Cells)

Synthesis: UBI-Ad

The N-terminal Fmoc-group of on-resin UB129-41 (15 μmol), synthesised by standard SPPS was removed by bubbling of the resin in 20% piperidine in DMF (2 ml). After washing, Fmoc-L-Lys(Fmoc)-OH (36 mg, 60 μmol), PyBOP (31 mg, 60 μmol), 1-Hydroxybenzotriazole (8.1 mg, 60 μmol) and DiPEA (20 μl, 120 μmol) were added in DMF (2 ml) and the suspension was mixed for 2 hours at room temperature. After washing steps with DMF/DCM and removal of the Fmoc-group, Fmoc-Gly-OH (36 mg, 120 μmol), PyBOP (62 mg, 120 μmol), 1-Hydroxybenzotriazole (16 mg, 120 μmol) and DiPEA (20 μl, 240 μmol) were added and again the suspension was mixed for 2 hours at room temperature. After washing steps with DMF/DCM and removal of the Fmoc-group, 1-adamantanecarbonylchloride (24 mg, 120 μmol), 1-Hydroxybenzotriazole (16 mg, 120 μmol) and DiPEA (40 μl, 240 μmol) were added and the mixture was stirred for 14 hours. After washing, the compound was cleaved off of the resin by stirring in a 38:1:1 TFA/TIPS/H2O solution for 2 hours. The remaining solution was precipitated in cold 1:1 MTBE/Hexane and the precipitate was washed. The resulting white solid was desiccated, purified using reversed-phase HPLC and lyophilised subsequently. MALDI-TOF calculated: 2258.7, found 2259.9.

Synthesis of Cy5_{0.5}CD₁₀PIBMA₃:

The synthesis of Cy5_{0.5}CD₁₀PIBMA₃₉ was performed as follows: Poly(isobutylene-alt-maleic anhydride) M_{w} 6,000 (30 mg, 5.0 μmol, Sigma-Aldrich) and Cy5-(SO₃)Sulfonate-(SO₃)Amine (5.0 mg, 5.6 μmol) were dissolved in 3 mL dry DMSO and N,N-diisopropylethylamine (DIPEA, 50 μL, 250 μmol Sigma-Aldrich) was added. After stirring at 80° C. for 7 h, 6-monodeoxy-6-monoamino-β-cyclodextrin (95 mg, 80 μmol, Cyclodextrin Shop) was added and the reaction mixture was left to stir for another 72 h at 80° C. After cooling to RT, the polymer was first dialyzed against H₂O for 1 day, then against 100 mM phosphate buffer pH 9.0 for 24 h, and subsequently against H₂O for another 5 days, while refreshing the dialysis medium daily. The solution was lyophilized to give a blue powder (87 mg, 5 μmol) and was stored at −20° C. Before usage, a small amount was aliquoted in PBS at 1 mg/mL concentration and stored (<one month) at 7° C.

Functionalization of Staph. aureus with UBI-adamantane (S. aureus-Ad₂): Staphylococcus aureus (ATCC 25922, cultured for 24 h in brain-heart-infusion broth) containing about 3×10⁹ viable bacteria were stored in Eppendorf tubes at −20° C. until further use. For functionalization, one portion was defrosted, washed 3 times (4 min×3,500 rpm) in PBS and 20 μL of UBI-Ad (1 mM in PBS) was added to 1 mL of the bacteria suspension. After agitation in a shaking water bath for 1 h at 37° C., the solution was washed 2 times with phosphate buffered saline (PBS) by 2 centrifugation steps (4 min×3,500 rpm). The obtained S. aureus-UBI-Ad was diluted in 1 mL of PBS (containing 2×10⁸ viable bacteria). For dual-isotope studies S. aureus bacteria were functionalised with ^{99m}Tc-labeled UBI-Ad identical according to the protocol as described above. Radiolabelling with technetium-99m was performed as described below for Cy5_{0.5}CD₁₀PIBMA₃₉. In these studies both the localization of bacteria in infections can be assessed with radio-imaging as well as the bacterial targeting.

Radiolabelling of Cy5_{0.5}CD₁₀PIBMA₃₉

Radiolabeling of Cy5_{0.5}CD₁₀PIBMA₃₉ was performed as follows: to 10 μL of Cy5_{0.5}CD₁₀PIBMA₃₉ (1 mg/mL PBS), 4 μL of SnCl₂.2H₂O (0.44 mg/mL saline, Technescan PYP, Mallinckrodt Medical B.V.), and 100 μL of a freshly eluted ^{99m}Tc-Na-pertechnetate solution (500 MBq/mL, Mallinckrodt Medical B.V.) were added and the mixture was gently stirred in a shaking water bath for 1 h at 37° C., as described in M. M. Welling, A. Paulusma-Annema, H. S. Balter, E. K. J. Pauwels and P. H. Nibbering, Eur. J. Nucl. Med., 2000, 27, 292-301. Subsequently, the labelling yield was estimated over time by ITLC analysis according the following procedure: 2 μL of the reaction mixture was applied on 1×7 cm ITLC-SG paper strips (Agilent Technologies, USA) for 10 min at room temperature with acetone as mobile phase. After 1 h the highest labelling yield of Cy5_{0.5}CD₁₀PIBMA₃₉ with technetium-99m was assessed (49.6%±12.8) and the reaction mixture was purified by size exclusion chromatography with sterile PBS as mobile phase using Sephadex™ G-25 (desalting columns PD-10, GE Healthcare Europe GmbH, Freiburg, Germany). Fractions containing ^{99m}Tc-Cy5_{0.5}CD₁₀PIBMA₃₉ were collected and directly applied in the imaging experiments. According the data calculated from the PD-10 purification a labelling yield of 49.2%±6.9 was obtained, which was in accordance with the yield estimated by ITLC analysis. For dual isotope imaging, Cy5_{0.5}CD₁₀PIBMA₃₉ was labeled with indium-111 as follows: to 10 μL of Cy5_{0.5}CD₁₀PIBMA₃₉ (1 mg/mL PBS), 40 μL of 0.25 M NH₄-acetate pH 5.5, and 30-50 μL of a InCl₃ solution (111 MBq/0.3 mL, Mallinckrodt Medical B.V.) were added and the mixture was gently stirred in a shaking water bath for 1 h at 37° C. Radiochemical analysis was performed as described herein-above.

Stability of Radiolabelled Cy5_{0.5}CD₁₀PIBMA₃

To assess the stability of the radiolabelling, after 24 h the release of radioactivity from PD-10 purified ^{99m}Tc-Cy5_{0.5}CD₁₀PIBMA₃₉ or ¹¹¹In-Cy5_{0.5}CD₁₀PIBMA₃₉ was determined with ITLC (according the same methods as described herein), Release was less than 5% of the total radioactivity.

Supramolecular Interaction Between S. aureus-Ad₂ and ^{99m}Tc-Cy5_{0.5}CD₁₀PIBMA₃₉

To determine the supramolecular interaction between UBI-Ad and Cy5_{0.5}CD₁₀PIBMA₃₉ in vitro, 0.1 mL of UBI-Ad in PBS (0.2 mg/mL) and 0.1 mL of ^{99m}Tc-Cy5_{0.5}CD₁₀PIBMA₃₉ or ¹¹¹InCy5_{0.5}CD₁₀PIBMA₃₉ in PBS (1 mg/mL, 1 MBq) were mixed and the solution was incubated for 1 h in a shaking water bath at 37° C. Thereafter, the radioactivity of the total amount added and the radioactivity of the pellet after two washing steps with PBS were measured in a dose-calibrator, to determine the amount of binding of radiolabelled Cy5_{0.5}CD₁₀PIBMA₃₉ to UBI-Ad. After correction for background activity the amount of binding was expressed as the percentage of the total amount of radioactivity (% binding). To assess the effect of the Ad moieties, the same experiment was also performed with non-functionalized bacteria and the resulting % binding of radiolabelled Cy5_{0.5}CD₁₀PIBMA₃₉ to non-functionalized bacteria and S. aureus-Ad were compared. Compared to the binding to non-functionalized bacteria, a significant (p<0.01) higher binding of radiolabelled Cy5_{0.5}CD₁₀PIBMA₃₉ to S. aureus-Ad was calculated (using a two-tailed student t-Test, n=4). The supramolecular interaction between S. aureus-Ad and Cy5_{0.5}CD₁₀PIBMA₃₉ was also visualized by confocal microscopy, employing the Cy5 component of the polymer. For this purpose, the same experiment was repeated, but this time non-radioactive Cy5_{0.5}CD₁₀PIBMA₃₉ was added to the non-functionalized bacteria and S. aureus-UBI-Ad solutions. After washing, 10 μL of bacteria (with or without UBI-Ad₂) Cy5_{0.5}CD₁₀PIBMA₃₉ solution was pipetted onto culture dishes with glass insert (035 mm glass bottom dishes No. 15, poly-d-lysine coated, γ-irradiated, MatTek corporation). Images were taken on a Leica SP5 WLL confocal microscope under 63× magnification using Leica Application Suite software. Cy5 fluorescence was measured with excitation at 633 nm, emission was collected at 650-700 nm.

Animals

All in vivo studies were performed using 2-3 month old Swiss mice (20-25 g, Crl:OF1 strain, Charles River Laboratories, USA). All animal studies were approved by the institutional Animal Ethics Committee (DEC permit 12160) of the Leiden University Medical Center. All mice were kept under specific pathogen-free conditions in the animal housing facility of the LUMC. Food and water were given ad libitum.

Pathogen Inoculation

Animals were inoculated using an intra muscular injection of 0.1 mL S. aureus-UBI-Ad₂ (2×10⁸ viable bacteria/mL) in the right thigh muscle. After 1 hr 0.1 mL ^{99m}Tc-Cy5_{0.5}CD₁₀PIBMA₃₉ (10-20 MBq per mouse) was injected into a tail vein. For dual isotope imaging, ^{99m}Tc-S. aureus-Ad and In-Cy5_{0.5}CD₁₀PIBMA₃₉ were injected under the same conditions.

General SPECT Imaging and Biodistribution Protocol

SPECT imaging was performed as follows: at 2 h after injection of ^{99m}Tc-labeled compounds mice were placed and fixed onto a dedicated positioned bed of a three-headed U-SPECT-2 (MILabs, Utrecht, The Netherlands) under continuous 1-2% isoflurane anesthesia. Radioactivity counts from total body scans or selected regions of interest (ROI) were acquired for 60 min using a 0.6 mm mouse multi-pinhole collimator in list mode data. For reconstruction from list mode data, the photo peak energy window was centered at 140 keV with a window width of 20%. Side windows of 5% were applied to correct for scatter and down scatter corrections. The image was reconstructed using 16 Pixel based Ordered Subset Expectation Maximization iterations (POSEM) with 6 subsets, 0.2 mm isotropic voxel size and with decay and triple energy scatter correction integrated into the reconstruction with a post filter setting of 0.25 mm, as described in W. Branderhorst, B. Vastenhouw and F. J. Beekman, Phys. Med. Biol., 2010, 55, 2023-2034.

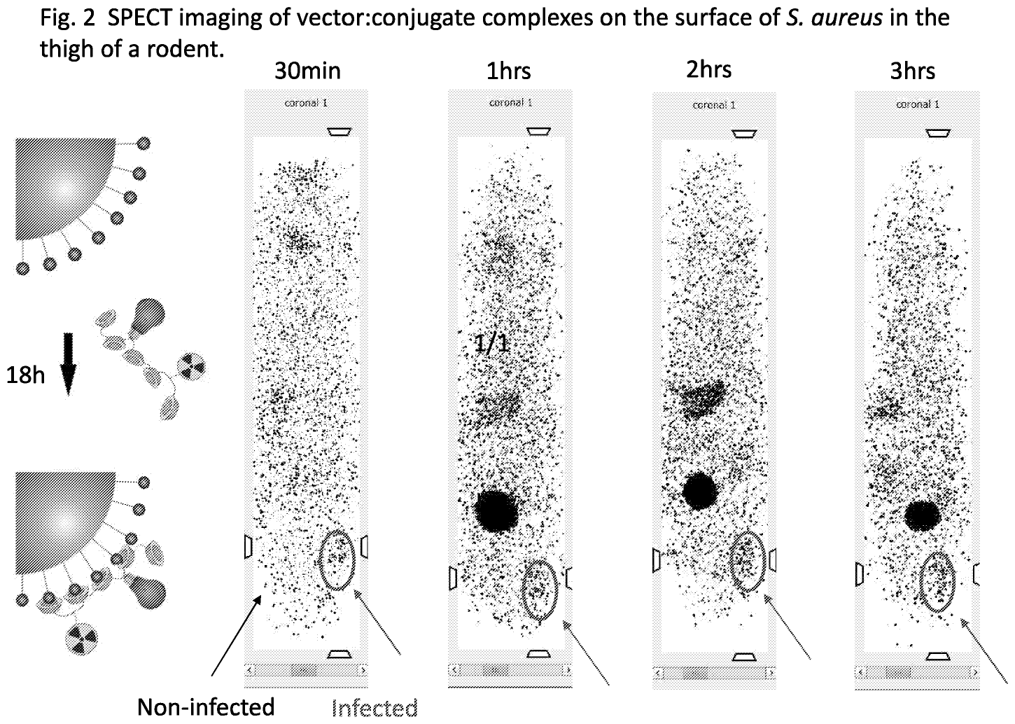

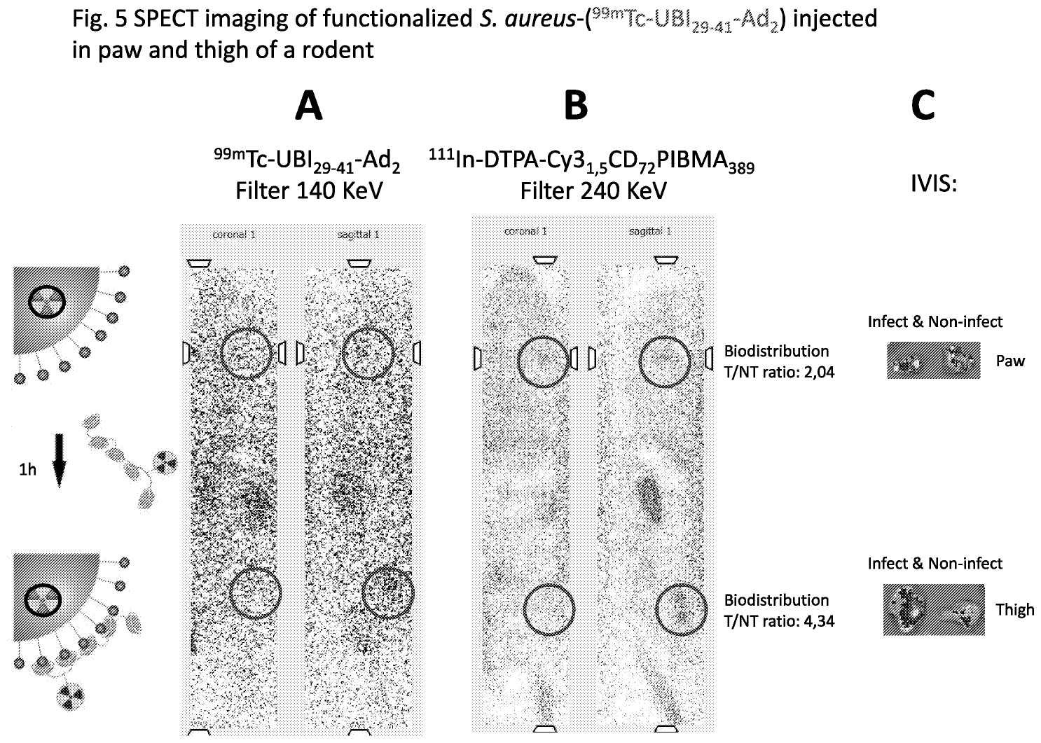

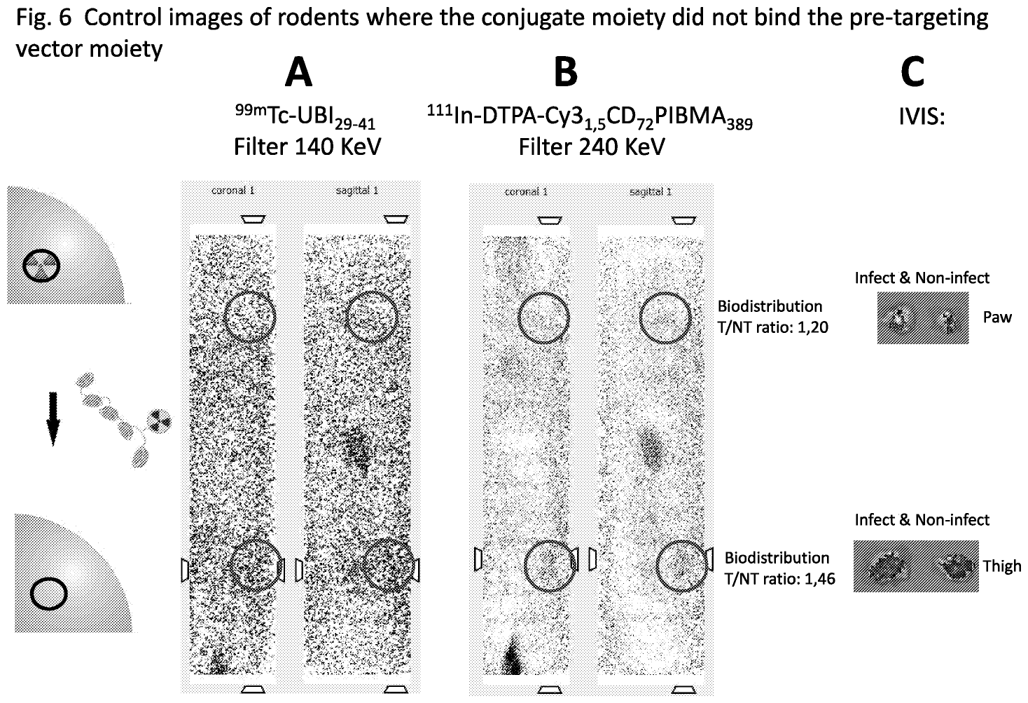

Volume-rendered images were generated from 2-4 mm slices and analyzed using Matlab R2014a software (version 8.3.0.532, MathWorks® Natick, Mass.). Images were generated from maximum intensity protocols (MIP) adjusting the color scale threshold to optimal depiction of the tissues of interest, as set out in M. N. van Oosterom, R. Kreuger, T. Buckle, W. A. Mahn, A. Bunschoten, L. Josephson, F. W. B. van Leeuwen and F. J. Beekman, EJNMMI Res, 2014, 4, 56-56. After imaging, the mice were euthanized by an intraperitoneal injection of 0.25 mL Euthasol (ASTfarma, Oudewater, The Netherlands) and the biodistribution was carried out as described below. For dual isotope labeling, imaging was performed as describe above. Counts were collected within the 1-1200 KeV frame and thereafter, for image reconstruction, photo peak energy windows were centered at 140 keV (^{99m}Tc) or 240 keV (¹¹¹In). Side windows were applied on the other peaks to shield for the other isotope. FIG. 2 shows SPECT imaging of vector:conjugate complexes on the surface of S. aureus in the thigh of a rodent, while FIGS. 5 and 6 show dual-isotope microSPECT images of mice containing ^{99m}Tc-vector (S. aureus-^{99m}Tc-UBI-Ad; encircled) and ¹¹¹In-conjugate and dual-isotope microSPECT image of mice containing ^{99m}Tc-control (S. aureus-^{99m}Tc-UBI; encircled) and ¹¹¹In-conjugatem, respectively.

General Fluorescence Imaging and Biodistribution Protocol

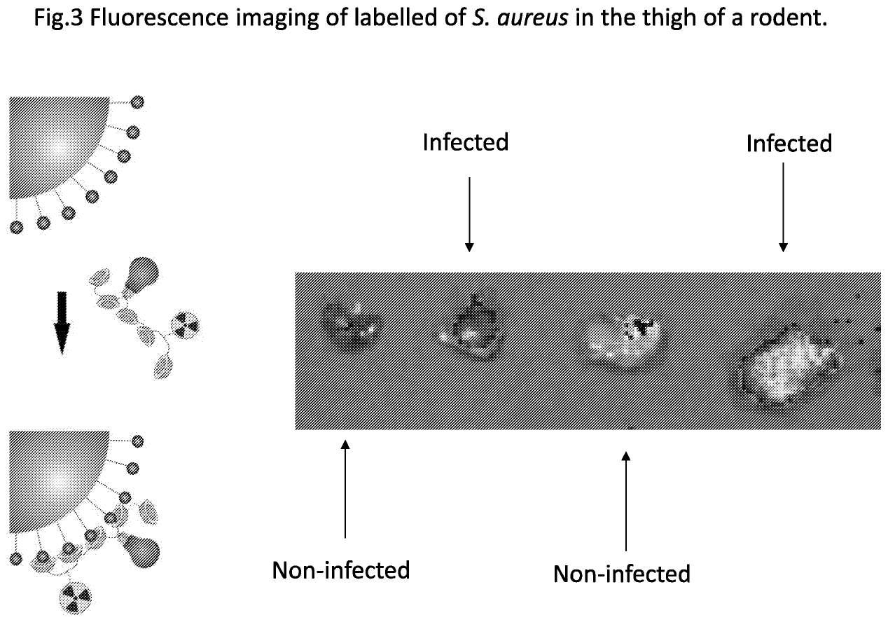

Fluorescence imaging of the injected mice was performed using the IVIS Spectrum imaging system (Caliper Life Science, Hopkinton, Mass.). Images were acquired following excitation at 640 nm, and light was collected >680 nm (acquisition time 5 s). Imaging analysis of the IVIS Spectrum data was performed using the Living Image software from xenogeny v 3.2 (Caliper LS). Thereafter, the various organs and/or injection sites were removed and imaged with the IVIS Spectrum. Since the fur caused additional attenuation of the fluorescent emission, it was removed from the muscles to allow for more detailed fluorescence imaging. Finally, fluorescent ex vivo imaging of excised muscles and other tissues was performed. FIG. 3 shows the fluorescence imaging of labelled of S. aureus in the thigh of a rodent.

Biodistribution

After SPECT imaging (as described above), organs and tissues were surgically removed and radioactivity was counted using a gamma counter (2470 automatic gamma counter, Perkin-Elmer, 245 keV, 60 s). Counts per minute were converted into MBq and corrected for decay. The percentage of the injected dose per gram of tissue (% ID g-1) was calculated.

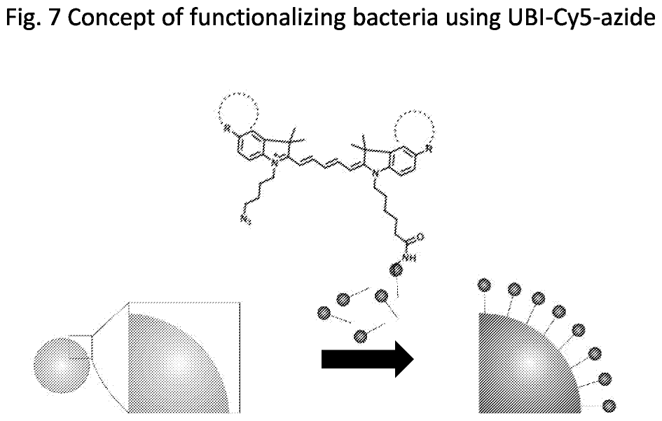

### Example 2: Concept of Functionalizing Bacteria Using UBI-Cy5-Azide (See FIG. 7)

All chemicals and solvents were obtained from commercial sources and used without further purification. HPLC was performed on a Waters HPLC system using a 1525EF pump and a 2489 UV/VIS detector. For preparative HPLC a Dr. Maisch GmbH Reprosil-Pur 120 C18-AQ 10 μm (250×20 mm) column was used (12 mL min⁻¹) and for semi-preparative HPLC a Dr. Maisch GmbH Reprosil-Pur C18-AQ 10 μm (250×10 mm) column was used (5 mL min⁻¹). Analytical HPLC was performed using a Dr. Maisch GmbH Reprosil-Pur C18-AQ 5 μm (250 4.6 mm) or a Dr. Maisch GmbH Reprosil-Pur C18-AQ 5 μm (250×10 mm) column while a gradient of 0.1% TFA in H₂O/CH₃CN 95:5 to 0.1% TFA in H₂O/CH₃CN 5:95 in 40 minutes (1 mL min⁻¹) was employed. Mass spectrometry was performed on a Bruker microflex MALDI-TOF using α-Cyano-4-hydroxycinnamic acid as matrix with granuliberin R as internal standard. UPLC/MS was performed on a Waters Acquity UPLC-MS system using a Acquity UPLC photodiode array detector, a SQ Detector mass spectrometer. Here a flow rate of 0.5 mL/min was used (Waters BEH C18 130 Å 1.7 μm (100×2.1 mm) column). NMR spectra of the new dye and phthalimidopropyl-sulfoindolenine were obtained with a Bruker AV-400 or 500 spectrometer (400 MHz ¹H NMR or 500 MHz ¹H NMR, respectively) and the chemical shifts (ppm (δ)) were related against tetramethylsilane (TMS). Abbreviations used include singlet (s), doublet (d), doublet of doublets (dd), triplet (t) and unresolved multiplet (m). Absorption spectra were recorded using a Ultrospec 3000 spectrometer (Amersham pharmacia biotech), from which a solvent blank was subtracted. Fluorescence measurements were performed using a Perkin-Elmer LS 55 fluorescence spectrometer that was equipped with a red-sensitive PMT. The fluorescence properties of dyes were determined according to published procedures.[21] Chloromethyl polystyrene resin (1% DVB, 200-400 mesh, 1.6-1.8 mmol/g) was obtained from TC chemicals. Cy7-DBCO quencher was purchased from Jena Bioscience, Germany.

### Synthesis of 3-Phthalimidopropyl trimethylindolenine

A mixture of potassium trimethylsulfoindolenine (1.5 g, 5.4 mmol), 3-bromopropyl phthalimide (4.5 g, 16.8 mmol, 3 equiv.) and tetrabutylammonium iodide (0.27 mmol, 0.05 equiv.) in 1,2-dichlorobenzene (15 ml) was heated to 100° C. for 18 hours, followed by 3 hours at 150° C. The crude indolenine slurry was subsequently precipitated in Et₂O (250 mL). The majority of the supernatant was removed by pipette, and from the remaining suspension the precipitate was collected by centrifugation (2000 rpm) and washed with Et₂O twice. Initial purification of the crude building block was performed by silica column chromatography (10%->20% MeOH in CH₂Cl₂). Further purification of this material by silica column chromatography (25% MeOH in EtOAc) and precipitation from Et₂O yielded the title compound as a pale yellow solid (0.8 g, 1.8 mmol; 34% yield). Rf (25% MeOH in EtOAc)=0.38. MALDI-TOF-MS: calculated m/z for [M+H]⁺ C₂₂H₂₃N₂O₅S=427.13, found 426.65.

¹H NMR (400 MHz, CD₃OD) δ=7.93-7.73 (m, 4H, Phth-Ar—H), 7.65-7.59 (m, 1H, Ar—H), 7.56 (d, J=1.7 Hz, 1H, Ar—H), 6.64 (d, J=8.3 Hz, 1H, Ar—H), 3.74 (t, J=7.4 Hz, 2H, N—CH₂—), 3.67 (t, J=7.1 Hz, 2H, N—CH₂—), 2.10-2.97 (m, 2H, CH₂—CH₂—CH₂), 1.34 (s, 6H, C—(CH₃)₂). The characteristic peak of the indolenine 2-methyl moiety was not observed, presumably due to proton exchange with the deuterated methanol.

### Synthesis of Cy5-(SO₃)Phthaimidyl-(SO3)COOH dye

Synthesis of Cy5-(SO₃)Phthalimidyl-(SO3)COOH was based on a slightly adapted compared to the procedure previously described by Lopalco et al. In short, carboxypentyl indolenine (940 mg, 2 mmol, 2 equiv.) and malonaldehyde dianil hydrochloride (540 mg, 2.2 mmol, 2.2 equiv.) were dissolved in 15 ml AcOH/Ac₂O (1:1 v/v) and subsequently heated to 120° C. for 2 hours. After cooling to room temperature, the now dark-colored mixture was precipitated in 300 ml Et₂O and the hemicyanine was collected as a brown precipitate. This was then washed once with Et₂O and twice with EtOAc. The crude hemicyanine was dissolved in DMF (50 mL) and added to previously prepared Merrifield resin-bound aminophenol (750 mg resin, 1 mmol amine moieties, 1 equiv.) in a 75 mL polypropylene vessel with frit and mixed using N₂ bubbling for 1 hour. The resin was repeatedly washed with DMF (50 mL) and CH₂Cl₂ (50 mL) until all the brown coloration was eluted. Next, 1 (210 mg, 0.5 mmol, 0.5 equiv.) and pyridine/Ac₂O (12 mL, 3:1 v/v) were then added to the washed resin and the suspension was mixed for 2 hours. The resulting deep blue filtrate was separated from the beads by filtration and the beads were washed once more with DMF. The collected filtrate fractions were combined and precipitated from Et₂O (300 mL) to obtain the crude dye as a dark blue solid (250 mg) that was used directly in the next reaction step. A small amount was purified by preparative HPLC for analysis. Analytical HPLC t_{R}=29.9 min. MALDI-TOF-MS: calculated m/z for [M+H]⁺ C₄₂H₄₆N₃O₁₀S₂=816.26, found 816.00. 1H NMR (400 MHz, d6-DMSO) δ=8.30-8.43 (m, 2H, cyanine bridge CH—CH—CH—CH—CH—CH), 7.92-7.79 (m, 6H, 4×phthalimide C—H+aryl), 7.59-7.67 (m, 2H, aryl), 7.33-7.41 (m, 2H, aryl), 6.48 (t, J=12.4 Hz, 1H CH—CH—CH—CH—CH—CH), 6.24-6.38 (m, 2H, cyanine bridge CH—CH—CH—CH—CH—CH), 4.22 (broad t, 2H, N—CH₂—CH₂—CH₂—NPhth), 4.12 (broad t, J=6.8 Hz, 2H, N—CH₂—CH₂—CH₂—CH₂—CH₂—COOH), 3.71 (t, J=7.2 Hz, 2H, N—CH₂—CH₂—CH₂—NPhth), 2.21 (t, J=7.2 Hz, 2H, N—CH₂—CH₂—CH₂—CH₂—CH₂—COOH), 2.00- 2.10 (m, 2H, N—CH₂—CH₂—CH₂—NPhth), 1.71 (2×s+m, 14H, 4×indolenine CH₃+N—CH₂—CH₂—CH₂—CH₂—CH₂—COOH), 1.62-1.48 (m, 2H, N—CH₂—CH₂—CH₂—CH₂—CH₂—COOH), 1.33-1.43 (m, 2H, N—CH₂—CH₂—CH₂—CH₂—CH₂—COOH) ppm.

### Synthesis of Cy5-(SO₃)amine-(SO₃)COOH Dye

100 mg of 2 was dissolved in a methylamine solution (33% in EtOH, 8 mL), to which water (1 mL) was added. The solution was allowed to stir overnight, yielding a golden brown solution. Excess methylamine was removed using N₂ bubbling, followed by rotary evaporation. The now blue residue was dissolved in MeOH and precipitated in Et₂O. The blue precipitate was collected by precipitation and was purified using preparative HPLC. Following MS analysis, the fractions containing product were pooled and lyophilized to yield the title compound as a blue solid (25 mg, 36 μmol, 18% yield over two steps from the dye building blocks). Analytical HPLC t_{R}=24.6 min. MALDI-TOF-MS, calculated for [M+H]⁺ C₃₄H₄₄N₃O₈S₂=686.26, found 686.43. ¹H NMR (500 MHz, d6-DMSO) δ=8.34-8.46 (m, 2H, cyanine bridge CH—CH—CH—CH—CH—CH), 7.85 (2×d J=11.4 Hz, 2H, aryl), 7.67-7.74 (broad m, 2H, NH₂), 7.63-7.67 (m, 2H, aryl), 7.33-7.41 (m, 2H, aryl), 6.57 (t, J=12.3 Hz, 1H, cyanine bridge CH—CH—CH—CH—CH—CH), 6.26-6.41 (2×d, J=13.8 Hz, 2×1H, cyanine bridge CH—CH—CH—CH—CH—CH), 4.11-4.21 (m, 4H, N—CH₂—CH₂—CH₂—NH₃+N—CH₂—CH₂—CH₂—CH₂—CH₂—COOH), 2.86-2.95 (m, 2H, N—CH₂—CH₂—CH₂—NH₂), 2.21 (t, J=7.2 Hz, 2H, N—CH₂—CH₂—CH₂—CH₂—CH₂—COOH), 1.90-2.04 (m, 2H, N—CH₂—CH₂-CH₂—NH₂), 1.71 (2×s+m, 14H, 4×indolenine CH₃+N—CH₂—CH₂—CH₂—CH₂—CH₂—COOH), 1.50-1.60 (m, 2H, N—CH₂—CH₂—CH₂—CH₂—CH₂—COOH) 1.32-1.42 (m, 2H, N—CH₂—CH₂—CH₂—CH₂—CH₂—COOH) ppm.

### Synthesis of N₃-Cy5-COOH

3 (25.0 mg, 36 μmol) was dissolved in a mixture of H₂O (3 mL) and MeCN (1 mL) and the pH was adjusted to approximately 8 using NMM. A catalytic amount of CuSO₄ (1 mg) was added, followed by imidazolium azidosulfonyl chloride [23] (15 mg, 72 μmol, 2 equiv.). The reaction mixture was stirred for 2 hour after which a second portion of imidazolium azidosulfonyl chloride (5.0 mg, 24 μmol, 0.7 equiv.) was added and stirring was continued for 30 minutes. The reaction mixture was then purified using preparative HPLC after addition of KCl (20 mg) and subsequently lyophilized to yield the title product as a blue solid (9.0 mg, 12 μmol, 33% yield). Analytical HPLC t_{R}=29.1 min. MALDI-TOF-MS, calculated for [M+H]⁺ C₃₄H₄₄N₃O₈S₂=712.25, found 712.89. ¹H NMR (400 MHz, d6-DMSO, with added 4 mM Ethylene carbonate as internal standard [21]) δ=8.30-8.45 (m, 2H, cyanine bridge CH—CH—CH—CH—CH—CH), 7.83 (2×d J=9.1 Hz, 2H, aryl), 7.62-7.67 (m, 2H, aryl), 7.30-7.38 (m, 2H, aryl), 6.60 (t, J=12.3 Hz, 1H, cyanine bridge CH—CH—CH—CH—CH—CH), 6.26-6.42 (2×d, J=13.7 Hz, 2×1H, cyanine bridge CH—CH—CH—CH—CH—CH), 4.05-4.20 (m, 4H, N—CH₂—CH₂—CH₂—N₃+N—CH₂—CH₂—CH₂—CH₂—CH₂—COOH), 3.49 (under water peak, 2H, N—CH₂—CH₂—CH₂—N₃), 2.21 (t, J=7.2 Hz, 2H, N—CH₂—CH₂—CH₂—CH₂—CH₂—COOH), 1.90-2.05 (m, 2H, N—CH₂—CH₂—CH₂—N₃), 1.71 (2×s+m, 14H, 4×indolenine CH₃+N—CH₂—CH₂—CH₂—CH₂—CH₂—COOH), 1.50-1.60 (m, 2H, N—CH₂—CH₂—CH₂—CH₂—CH₂—COOH) 1.32-1.43 (m, 2H, N—CH₂—CH₂—CH₂—CH₂—CH₂—COOH) ppm.

Generation of N₃-Cy5-SPZ

Laboratory-reared Anopheles stephensi mosquitoes were infected with Plasmodium falciparum parasites (NF54) as described in Ponnudurai, T. et al. Infectivity of cultured Plasmodium falciparum gametocytes to mosquitoes. Parasitology 98 Pt 2, 165-173 (1989). Between 14-21 days after infection, salivary glands were dissected and collected in RPMI medium. Cy5-(SO₃)AzideC₃—(SO₃)COOH (57 nmol) in DMSO (5 μl) was added to a suspension salivary glands in PBS. The suspension was shaken for 3 hours at 37° C. after which it was centrifuged at 13000 RPM for 30 seconds. A blue pellet was visible and the supernatant was carefully removed. 150 μl of RPMI+10% FCS was added and the suspension was vortexed. Again the supernatant was removed and the process was repeated 2 more times. The salivary glands were crushed and the blue pellet was resuspended with 150 μl of RPMI+10% FCS and used in further experiments.

The generation of N₃-Cy5-sPf was also visualized by confocal microscopy, employing the Cy5 component. For this purpose, N₃—Cy5-sPf was pipetted onto culture dishes with glass insert (035 mm glass bottom dishes No. 15, poly-d-lysine coated, γ-irradiated, MatTek corporation). Images were taken on a Leica SP5 WLL confocal microscope under 63× magnification using Leica Application Suite software. Cy5 fluorescence was measured with excitation at 633 nm, emission was collected at 650-700 nm.

Photophysical Properties of N₃-Cy5-COOH with Cy7-DBCO Quencher

To a solution of N₃-Cy5-COOH (100 μM in PBS, 30 μL), Cy7-DBCO was added (800 μM in DMSO, 7.5 μL) and the resulting mixture was gently shaken for 60 minutes. The resulting solution was diluted 100-fold in DMSO or PBS and a 2D fluorescence heat map was generated, which was compared to freshly prepared mixtures of N₃-Cy5-COOH (1 μM in PBS or DMSO respectively) with Cy7-DBCO (800 μM in DMSO, 7.5 μL).

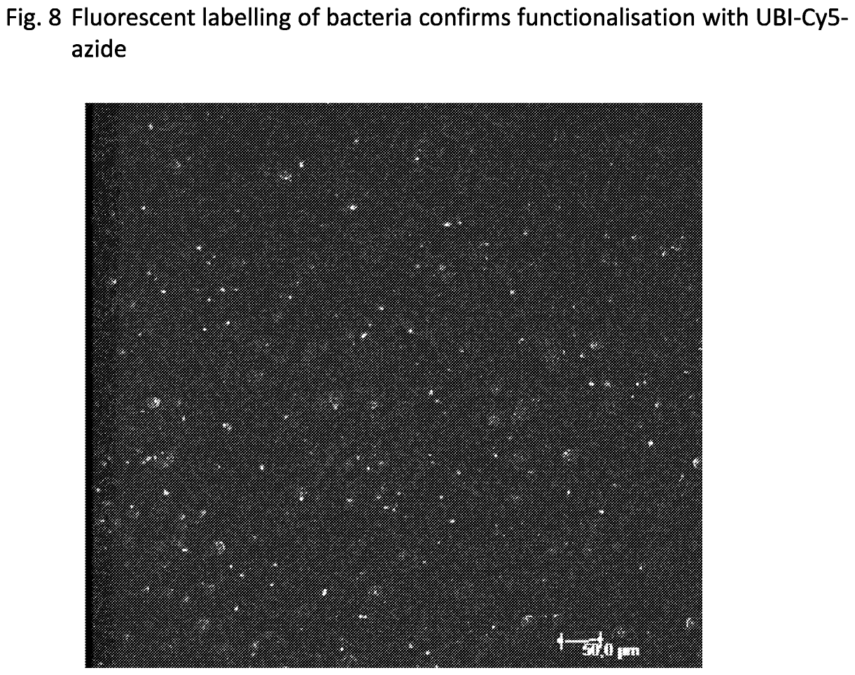

Absorbance spectra were also measured of the four samples. The quenching rate was also investigated by fluorescence measurements using solutions of N₃-Cy5-COOH (1 μM in PBS, 3 mL) after addition of Cy7-DBCO (800 μM in DMSO, 7.5 μL, 2 equiv.). Fluorescence and absorbance measurements were performed at 5-minute intervals. FIG. 8 shows the fluorescent labelling of bacteria confirms functionalisation with UBI-Cy5-azide.

Photophysical Properties of N₃-Cy5-sPf with Cy7-DBCO Quencher

Of the suspension containing N₃—Cy5-sPf 80 ul was pipetted in a cuvette and diluted 6.25 times by adding 420 μl of water. The fluorescence (excitation at 610 nm) was measured using a fluorescence spectrometer (Perkin Elmer LS-55). Following the addition of 0.4 nmol of Cy7-DBCO quencher in PBS (5 μl), the solution was homogenised and measured again immediately, followed by measuring at 10 min intervals until Cy5-fluorescence was (almost) zero.

UBI-Cy5-Azide Functionalized S. aureus

S. aureus bacteria (see Example 1) are functionalised as follows: 0.1 mL suspensions of S. aureus (containing 1×10⁶-1×10⁹ viable bacteria/mL PBS) are incubated for 1 h at room temperature with 10 μL UBI₂₉₋₄₁-Cy5-azide (1 μM) during gentle shaking. As a control, we incubated another batch of bacteria with 10 μL UB₂₉₋₄₁ (1 μM) under identical conditions. Thereafter, the bacteria are washed twice with PBS (4 min, 3,500 rpm).

The functionalization of bacteria with UBI-Cy5-azide was also visualized by confocal microscopy, employing the Cy5 component of the peptide. For this purpose, a UBI-Cy5-azide functionalized S. aureus solution was pipetted onto culture dishes with glass insert (ø35 mm glass bottom dishes No. 15, poly-d-lysine coated, γ-irradiated, MatTek corporation). Images were taken on a Leica SP5 WLL confocal microscope under 63× magnification using Leica Application Suite software. Cy5 fluorescence was measured with excitation at 633 nm, emission was collected at 650-700 nm.

The above examples show that the subject invention can be successfully employed for a wide range of multi- or singular cell pathogens.

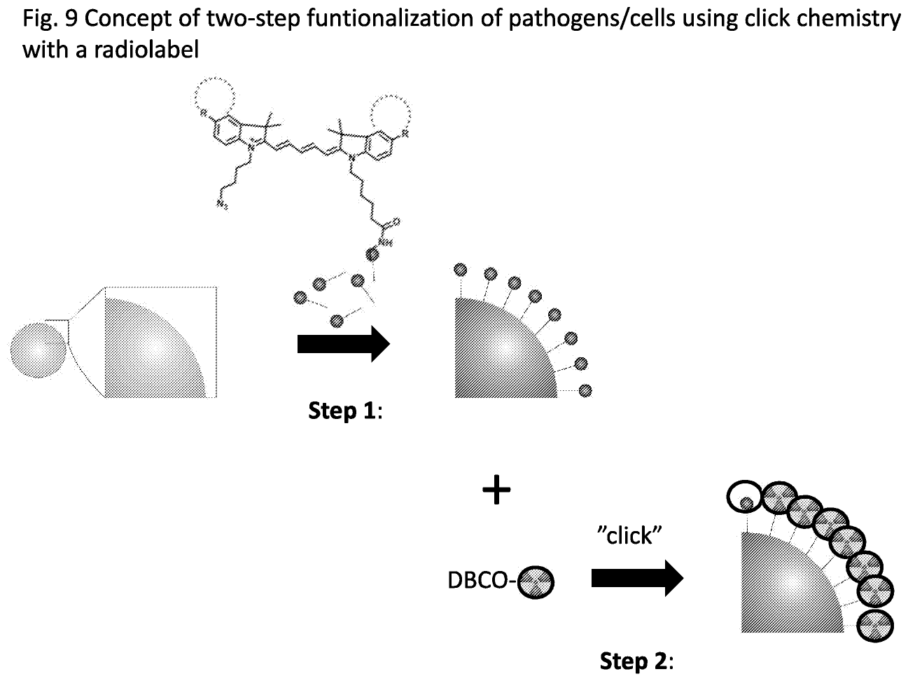

Radiolabelling of DBCO-DTPA (See FIG. 9 for the Two-Step Funtionalization of Pathogens/Cells Using Click Chemistry with a Radiolabel

For isotope labeling, DBCO-DTPA was labeled with indium-111 as follows: to 10 [symbol] L of DBCO-DTPA (1 mg/mL H₂O, 1.38 nM), 90 μL of 0.25 M NH₄-acetate pH 5.5, and 50 μL of a InCl₃ solution (111 MBq/0.3 mL, Mallinckrodt Medical B.V.) were added and the mixture was gently stirred in a shaking water bath for 1 h at room temperature.

Subsequently, the labeling yield was estimated over time by ITLC analysis according the following procedure: 2 μL of the reaction mixture was applied on 1×7 cm ITLC-SG paper strips (Agilent Technologies, USA) for 10 min at room temperature with 0.25 M NH₄-acetate pH 5.5 as mobile phase. After 1 h the highest labeling yield of DBCO-DTPA with indium-111 was assessed (>98%) and was directly applied in the experiments. To assess the stability of the radiolabeling, after 24 h the release of radioactivity from ¹¹¹In-DBCO-DTPA was determined with ITLC (according the same methods as described above), and this turned out to be less than 5% of the total radioactivity.

Labelling of UBI-Cy5-Azide S. aureus Functionalized With ¹¹¹In-DBCO-DTPA

UBI-Cy5-azide functionalized S. aureus (1×10⁶-1×10⁹ viable bacteria/mL PBS) in 1 mL are mixed with 15 μL of freshly labelled ¹¹¹In-DBCO-DTPA and incubated for 1 h or at 3 h at room temperature during gentle shaking. As a blank control, incubations without bacteria were performed. Thereafter, the bacteria are washed twice with PBS (4 min, 3,500 rpm) and the total incubation tube, the bacteria pellet, and the washing solutions are counted for radioactivity. For each sample of bacteria numbers, UBI peptide and time interval the binding of ¹¹¹In-DBCO-DTPA to the bacterial pellet was calculated and corrected for the blank.

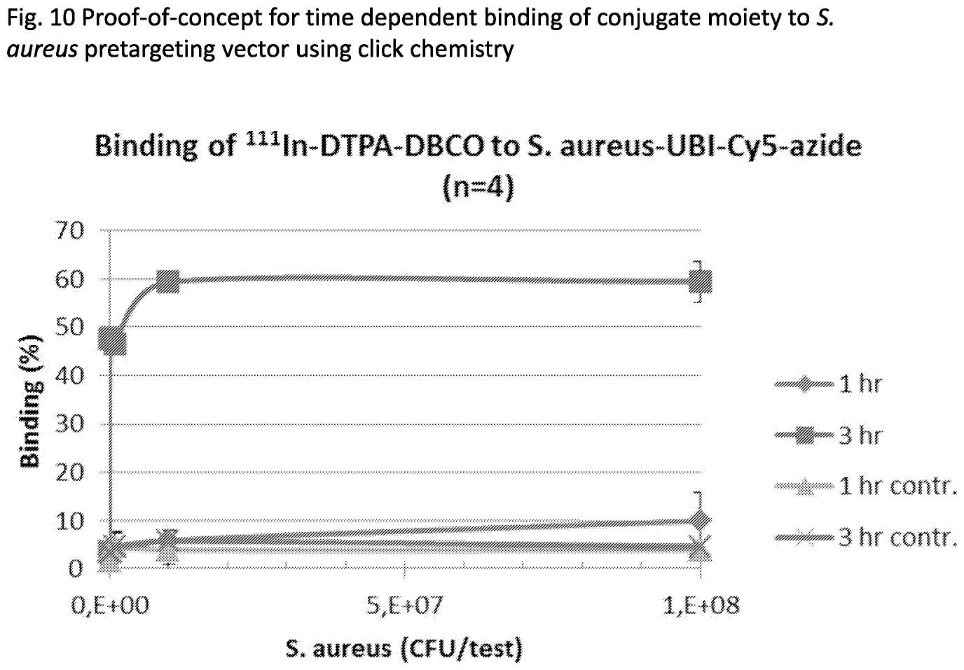

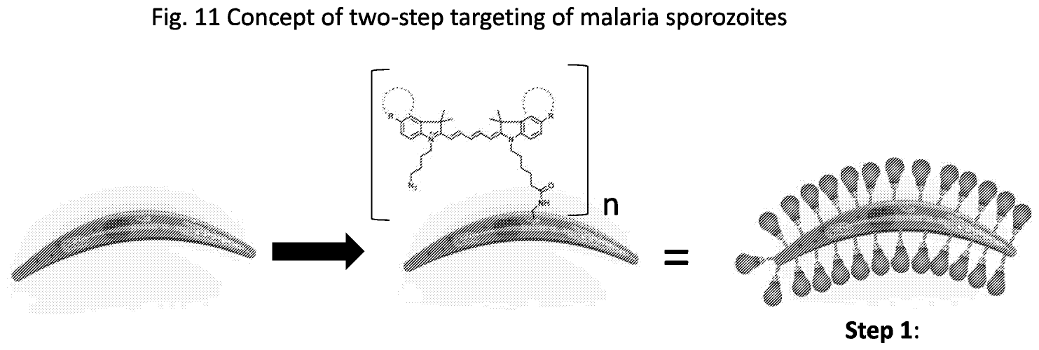

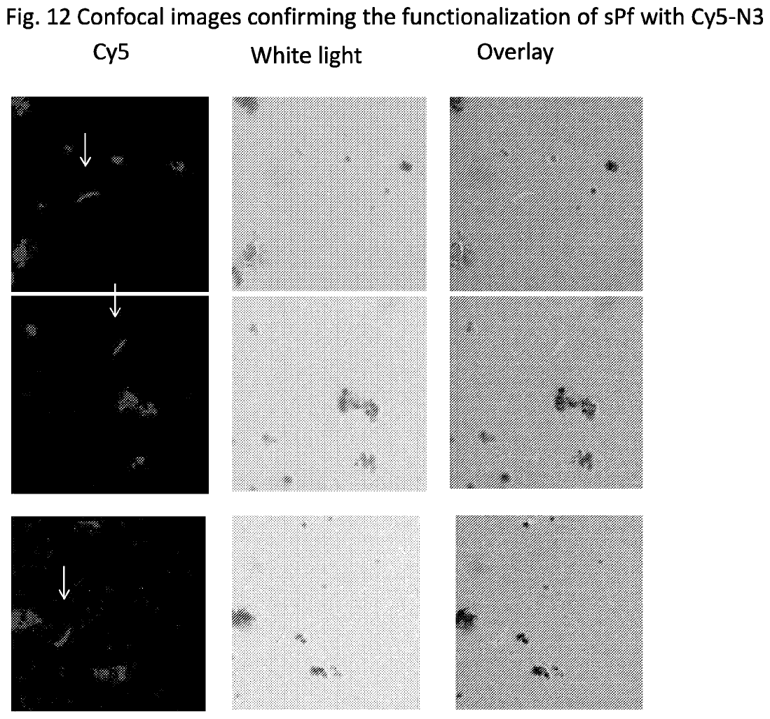

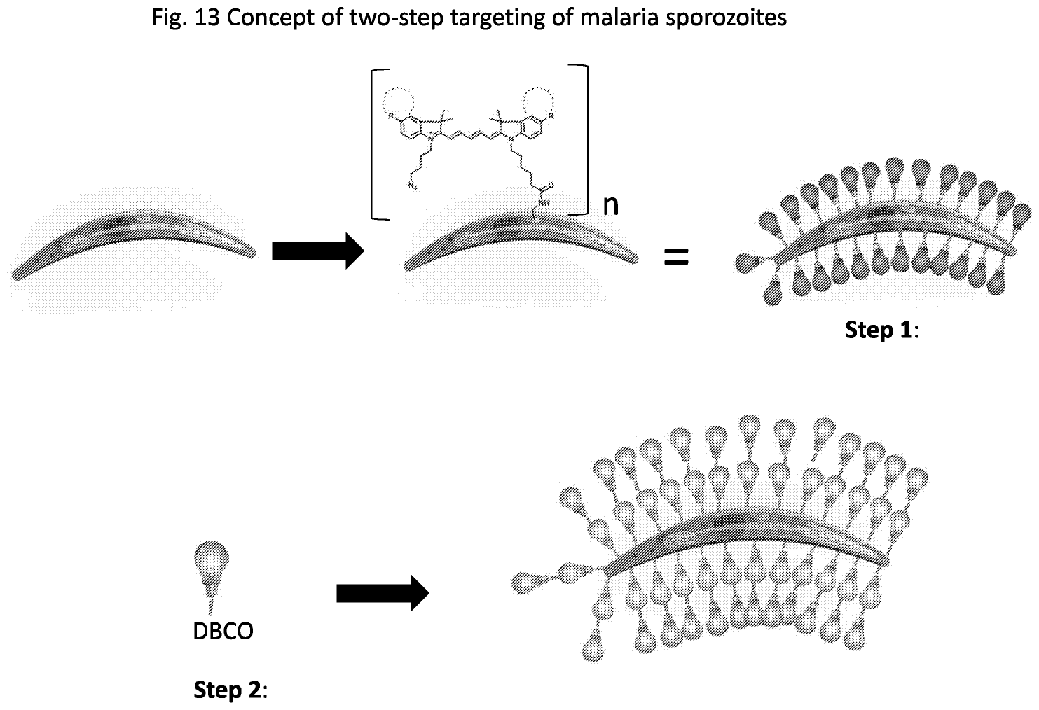

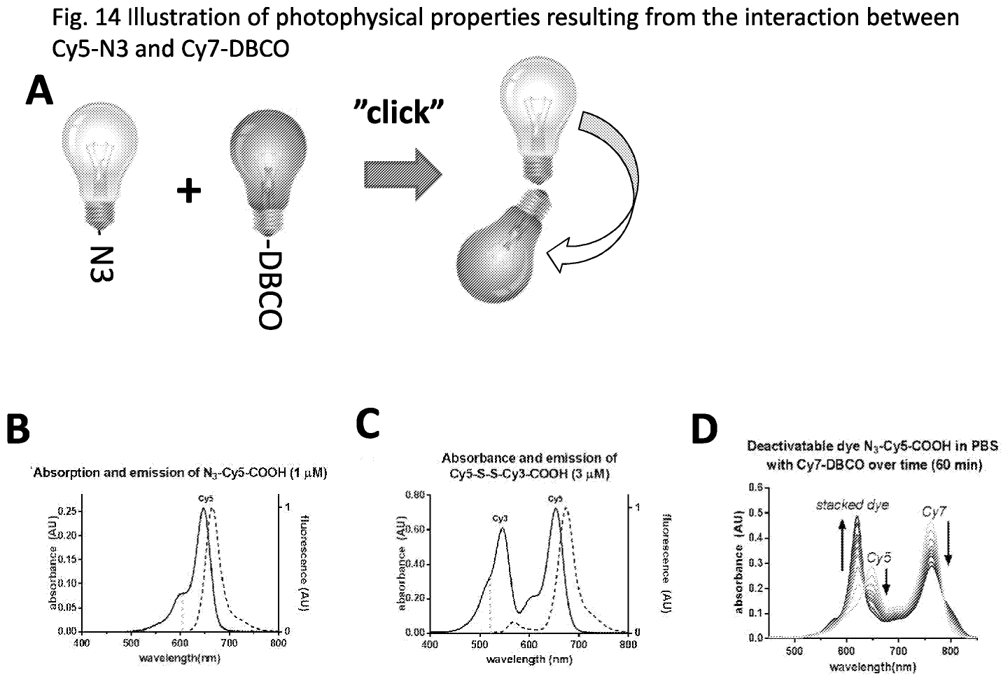

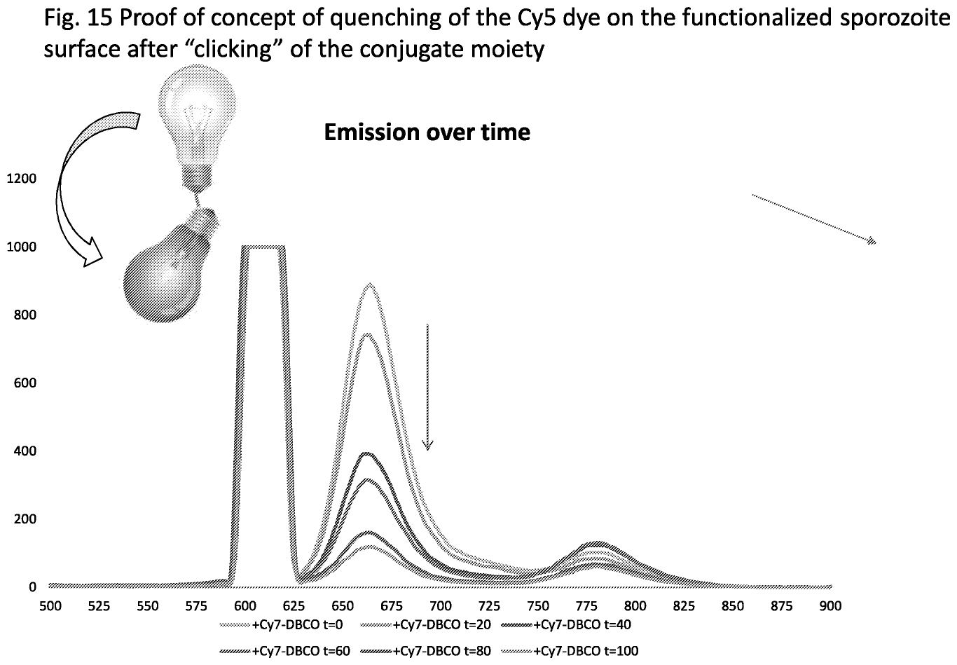

FIG. 10 shows the time dependent binding of conjugate moiety to S. aureus pretargeting vector using click chemistry.

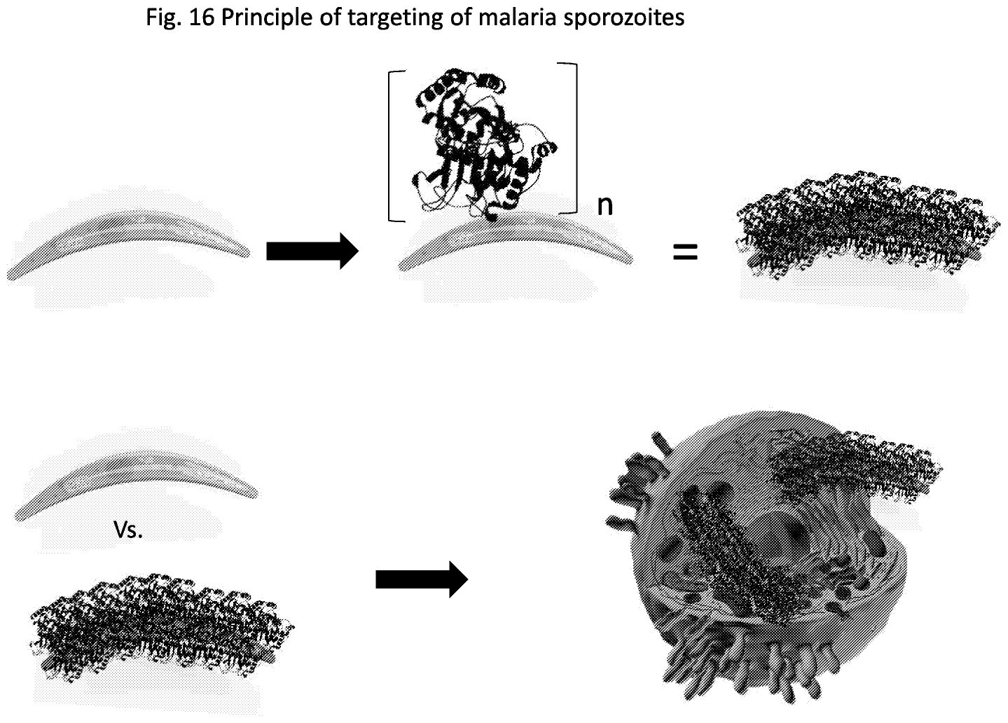

### Example 3: Sporozoite Interaction with Immune Cells (See FIG. 16 for an Illustration of the Concept)

Laboratory-reared Anopheles stephensi mosquitoes were infected with GFP-expressing Plasmodium berghei by feeding on mice infected with the parasite 3-4 days earlier. After 21-28 days, whole salivary glands were manually dissected, collected in RPMI medium and subsequently crushed and homogenized to free sporozoites from their glands. The free sporozoites were resuspended in 100 ul and counted in a Bürker counting chamber using phase-contrast microscopy.

Monocyte-derived dendritic cells (MoDC) and macrophages (MoMac) were obtained by culture of peripheral blood mononuclear cells according to previously described protocols. The MoDC and MoMac were incubated with genetically modified Plasmodium berghei sporozoites expressing GFP for 1 hour at 37 degrees 5% CO2 at a 1:1 ratio. Sporozoites were added either directly, or opsonized before stimulation by incubation with antiPbCS antibody (3D11, provided by Antonio Mendes, iMM, Lisbon) for 30 minutes at room temperature. After 1 hour of stimulation, cells were harvested and kept on ice to block further phagocytosis. Uptake of fluorescent sporozoites was measured by flow cytometry.

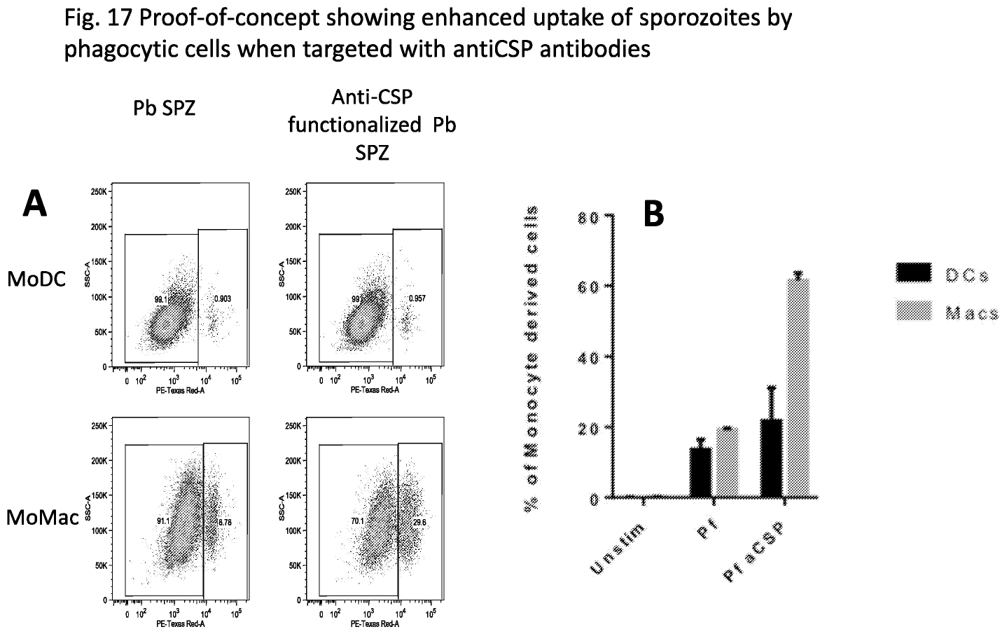

Percentage of opsonized cells was calculated by gating on GFP positive cells and diving the positive cells over the total number of counted cells by the flow cytometer. FIG. 17, Panel A shows a representative example of the gating strategy, FIG. 17 panel B shows the percentage of opsonized cells over all experiments. Monocyte derived dendritic cells are represented by black bars, monocyte derived macrophages are represented by gray bars. Whiskers represent standard deviations. Unstimulated cells were taken along as negative control.

The above examples illustrate that the subject invention can be successfully and broadly applied to a wide range of different pathogens.

1. A two component vaccine composition for use in in vivo administration, comprising a (preferably attenuated) pathogen or commensal modified as a pre-targeting vector, the pre-targeting vector comprising one or more pendent reactive moieties able to form a high affinity interaction with a complementary conjugate moiety residing on an immunogenic secondary component.

2. A The composition of claim 1, wherein the high affinity interaction comprises a supramolecular vector-conjugate interaction in an inclusion affinity complex.

3. The composition of claim 1, wherein a primary moiety on the vector surface is selected from the group consisting of streptavidin, cyclodextrin, antibodies, antibody fragments, ligands and aptamers, and wherein the conjugate moiety is a moiety complementary to the respective vector moiety.

4. The composition of claim 3, wherein the vector moiety comprises adamantane groups, and wherein the conjugate moiety includes cyclodextrin groups.

5. The composition of claim 1, wherein the high affinity interaction comprises formation of one or more covalent bonds.

6. The composition of claim 5, wherein the pathogen or commensal is functionalised to form a covalent click connection with a conjugate moiety, preferably using copper-free click chemistry.

7. The composition of claim 5, wherein the vector moiety comprises one or more azide groups, and wherein the conjugate moiety linked to the pathogen comprises one or more reactive alkyne groups, preferably wherein both groups are suited for copper-free click chemistry.

8. The composition of claim 1, wherein the composition comprises the vector component in an amount suitable to solicit a therapeutically effective immune response upon exposure to the conjugate moiety.

9. (canceled)

10. The composition of claim 1, wherein the pre-targeting vector moiety comprises a diagnostic label selected from the group consisting of: a diagnostic agent, an imaging agent, a contrast agent, a therapeutic agent, preferably a diagnostic label selected from the group consisting of magnetic resonance contrast labels, negative contrast labels, radioopaque contrast labels, ultrasound contrast labels, fluorescence labels, fluorescent contrast labels and (radio)isotope contrast labels, or combinations thereof.

11. The composition of claim 10, wherein the diagnostic label comprises a source of fluorescence radiation, or detectable gamma rays, preferably a medical radioisotope, more preferably ^{99m}Tc.

12. An immunogenic conjugate component for intravenous or local administration for forming a high affinity interaction with a complementary pre-targeting vector composition of claim 1, wherein the conjugate component comprises a complementary functionality for selectively coupling to the pre-targeting vector, and at least one agent selected from the group consisting of: a diagnostic agent, an imaging agent, a contrast agent, a therapeutic agent, or a combination or multitude thereof.

13. The component of claim 12, wherein the conjugating component a) is an immune enhancing agent such as a pathogen-associated molecular pattern, antigen, antibody, a target for pathogen recognition receptors or adjuvants, or b) comprises an antibody selective for the pathogen and known as immunogenic, preferably a circumsporozoite antibody for malaria sporozoites as pathogens.

14. (canceled)

15. The composition of claim 12, wherein the diagnostic agent is selected from the group consisting of magnetic resonance contrast agents, negative contrast agents, radioopaque contrast agents, ultrasound contrast agents, fluorescent contrast agent and (radio)isotope contrast agents, preferably radioisotopes, fluorescent dyes and fluorescent labels.

16. An enhanced vaccine composition comprising a) an immunogenic amount of a pathogen or commensal component modified as a pre-targeting vector of claim 1, that is immunogenically functional to generate immune responses directed against a pathogen or commensal upon conjugation, and b) a physiologically acceptable immunogenic conjugate component as an adjuvant comprising an effective amount of the immunogenic moiety, wherein the conjugate component comprises a complementary functionality for selectively coupling to the pre-targeting vector, and at least one agent selected from the group consisting of: a diagnostic agent, an imaging agent, a contrast agent, a therapeutic agent, or a combination or multitude thereof.

17. A method of stimulating an immune response in a human against pathogens or commensals, which comprises the steps of
a. administering to the human a functionalized pathogen or commensal modified as the pre-targeting vector component of claim 1, and
b. administering to the human a physiologically acceptable conjugate component, wherein the conjugate component comprises a complementary functionality for selectively coupling to the pre-targeting vector, and at least one agent selected from the group consisting of: a diagnostic agent, an imaging agent, a contrast agent, a therapeutic agent, or a combination or multitude thereof, inducing or adjuvanting, at the location of the pathogen or commensal an immune response vis-à-vis the pathogen or commensal.

18. The method of claim 17, comprising permitting the pre-targeting vector to reach or accumulate in the desired location before inducing or adjuvanting the immune response using the conjugate component.

19. The method of claim 17, wherein the pathogen or commensal is a pathogen selected from the group consisting of bacteria, rickettsia, mycoplasma, mycobacteria, protozoa, fungi, unicellular parasites, and multicellular parasites.

20. The method according to claim 19, wherein the human is suffering from infection by an infectious microorganism selected from the group consisting of bacteria, rickettsia, mycoplasma, mycobacteria, protozoa, fungi, and parasites, or wherein the human is suffering from a disbalanced microbiome caused by a commensal organism selected from the group consisting of bacteria and fungi.

21. (canceled)

22. The method of claim 19, wherein the human is not suffering from an infection, and wherein the immune response results in protective immunity against infection by an infectious agent exhibiting a targeted infectious agent marker, or against microbiome disturbances by inducing immunity against specific commensals.

23. The method of claim 17, further comprising providing a secondary conjugate component, wherein the secondary conjugate component is administered simultaneously or within a short period of time after administration of the primary conjugate component, to form an in-situ complex comprising the pre-targeting vector: primary conjugate: secondary conjugate.

24. The method of claim 23, wherein the secondary conjugate is administered before a pre-targeting vector: primary conjugate has been removed from a target cell surface.
